(19) United States
(12) Patent Application Publication
(10) Pub. No.: US 2025/0290409 A1
(43) Pub. Date: Sep. 18, 2025

(54) DRAINAGE HEIGHT IN PLACE EVALUATION FOR A WELL DRILLED INTO MULTIPLE FORMATION LAYERS

(71) Applicant: Chevron U.S.A. Inc., San Ramon, CA (US)

(72) Inventors: Chao Yan, Sugar Land, TX (US); Wei Wei, Sugar Land, TX (US); Wei Wang, Houston, TX (US)

(21) Appl. No.: 19/074,583

(22) Filed: Mar. 10, 2025

Related U.S. Application Data
(60) Provisional application No. 63/564,358, filed on Mar. 12, 2024.

(51) Int. Cl. E21B 49/00; G01N 33/28

(52) U.S. Cl. CPC E21B 49/005; G01N 33/2823

(57) ABSTRACT

A method for estimating a stimulated drainage height in place for a well drilled into multiple formation layers may include analyzing multiple batches of samples to generate associated measurements of parameters associated with a batch of samples, where the samples originate from a known range of depths in the well, and where the samples are collected at a surface outside the well. The method may also include generating a batch of signatures for each batch of samples using the associated measurements. The method may further include comparing the batches of signatures and generating a production allocation and a fluid correlation of the well. The method may also include generating an estimate of the stimulated drainage height in place for the well based on the production allocation and the fluid correlation of the well.

### CROSS-REFERENCE TO RELATED APPLICATIONS

This application claims priority to U.S. Provisional Patent Application Ser. No. 63/564,358 titled “Drainage Height In Place Evaluation For A Well Drilled Into Multiple Formation Layers” and filed on Mar. 12, 2024, the entire contents of which are hereby incorporated herein by reference.

### TECHNICAL FIELD

The present application is related to subterranean field operations and, more particularly, to stimulated drainage height in place (SDHIP) and extended drainage height in place (EDHIP) evaluations for a well drilled into multiple formation layers.

### BACKGROUND

Drainage height is used to estimate vertical fracture lengths that are active in transporting produced fluid for oil and gas production, such as in shale and tight (S&T) asset developments and reservoirs with tight formations in stacked plays. The vertical drainage heights (also sometimes referred to has lengths herein) may serve as channels to transport produced fluids to the surface. The efficiency of oil drainage from these channels is one of the factors related to reservoir and well performance.

### SUMMARY

In general, in one aspect, the disclosure relates to a method for estimating a stimulated drainage height in place for a well drilled into multiple formation layers The method can include analyzing a first plurality of samples to generate a first plurality of measurements of a plurality of parameters associated with the first plurality of samples, where the first plurality of samples originates from a first known range of depths in the well, and where the first plurality of samples is collected at a surface outside the well during a first period of time before production of the well. The method can also include generating a first plurality of signatures for the first plurality of samples using the first plurality of measurements. The method can further include analyzing a second plurality of samples to generate a second plurality of measurements of the plurality of parameters associated with the second plurality of samples, where the second plurality of samples originates from a known depth in the well, where the second subset of the plurality of samples is collected at the surface outside the well during a second period of time during production of the well, and where the second plurality of samples comprises produced fluid. The method can also include generating a second plurality of signatures for the second plurality of samples using the second plurality of measurements. The method can further include comparing the second plurality of signatures and the first plurality of signatures. The method can also include generating a production allocation and a fluid correlation of the well based on comparing the second plurality of signatures and the first plurality of signatures, where the production allocation includes a calibration matrix, and where the fluid correlation includes a quality assurance and quality control matrix. The method can further include generating an estimate of the stimulated drainage height in place for the well based on the production allocation and the fluid correlation of the well.

In another aspect, the disclosure relates to a data analysis system for estimating a stimulated drainage height in place for a well drilled into multiple formation layers. The system can include a plurality of sensor devices for measuring a plurality of parameters associated with a plurality of samples. The system can also include a controller communicably coupled to the plurality of sensor devices, where the controller is configured to analyze a first subset of a plurality of measurements taken by the plurality of sensor devices for a first subset of the plurality of samples, wherein the first subset of the plurality of samples originates from a first known range of depths in the well, and wherein the first subset of the plurality of samples is collected at a surface outside the well during a first period of time before production of the well. The controller can also be configured to generate a first plurality of signatures for the first subset of the plurality of samples using the first subset of the plurality of measurements. The controller can further be configured to analyze a second subset of the plurality of measurements taken by the plurality of sensor devices for a second subset of the plurality of samples, wherein the second subset of the plurality of samples originates from a second known range of depths in the well, and wherein the second subset of the plurality of samples is collected at a surface outside the well during a second period of time during production of the well. The controller can also be configured to generate a second plurality of signatures for the second subset of the plurality of samples using the second subset of the plurality of measurements. The controller can further be configured to compare the second plurality of signatures and the first plurality of signatures. The controller can also be configured to generate a production allocation and a fluid correlation of the well based on comparing the second plurality of signatures and the first plurality of signatures, where the production allocation includes a calibration matrix, and where the fluid correlation includes a quality assurance and quality control matrix. The controller can further be configured to generate an estimate of the stimulated drainage height in place for the well based on the production allocation and the fluid correlation of the well.

In yet another aspect, the disclosure relates to a computer implemented method for estimating a stimulated drainage height in place for a well drilled into multiple formation layers. The computer implemented method can include facilitate analyzing a first plurality of samples to generate a first plurality of measurements of a plurality of parameters associated with the first plurality of samples, where the first plurality of samples originates from a first known range of depths in the well, and where the first plurality of samples is collected at a surface outside the well during a first period of time before production of the well. The computer implemented method can also include facilitate generating a first plurality of signatures for the first plurality of samples using the first plurality of measurements. The computer implemented method can further include facilitate analyzing a second plurality of samples to generate a second plurality of measurements of the plurality of parameters associated with the second plurality of samples, where the second plurality of samples originates from a known depth in the well, where the second plurality of samples is collected at the surface outside the well during a second period of time during production of the well, and where the second plurality of samples comprises produced fluid. The computer implemented method can also include facilitate generating a second plurality of signatures for the second plurality of samples using the second plurality of measurements. The computer implemented method can further include facilitate comparing the second plurality of signatures and the first plurality of signatures, where the production allocation includes a calibration matrix, and where the fluid correlation includes a quality assurance and quality control matrix. The computer implemented method can also include facilitate generating a production allocation and a fluid correlation of the well based on comparing the second plurality of signatures and the first plurality of signatures. The computer implemented method can further include facilitate generating an estimate of the stimulated drainage height in place for the well based on the production allocation and the fluid correlation of the well.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, the same reference numerals used in different figures may designate like or corresponding but not necessarily identical elements.

FIG. 1 shows a field system in which example embodiments may be used.

FIG. 2 shows another field system in which example embodiments may be used.

FIGS. 3A and 3B show detailed views of the field system of FIG. 1 in which example embodiments may be used.

FIG. 4 shows a diagram of a system for SDHIP and EDHIP evaluation for a well drilled into multiple formation layers according to certain example embodiments.

FIG. 5 shows a system diagram of a controller according to certain example embodiments.

FIG. 6 shows a computing device in accordance with certain example embodiments.

FIG. 7 shows a flowchart of a method for SDHIP and EDHIP evaluation for a well drilled into multiple formation layers according to certain example embodiments.

FIGS. 8 through 11 show graphs that illustrate measurements of samples that are used to generate signatures for the samples according to certain example embodiments.

FIGS. 12 through 18 show examples of tables used to generate matrices of signatures for calibration and QA/QC according to certain example embodiments.

FIG. 19 shows a graph illustrating a calibration curve to estimate production allocation for a formation according to certain example embodiments.

FIG. 20 shows an example of a table used for QA/QC purposes according to certain example embodiments.

FIGS. 21 and 22 show examples of graphs illustrating production allocation for wells according to certain example embodiments.

FIG. 23 shows a graph of SDHIP and EDHIP ranges of a well 420 through several formation layers according to certain example embodiments.

FIG. 24 shows a gun barrel diagram of multiple wells in multiple formation layers for use with an estimated range of EDHIP with certain example embodiments.

### DETAILED DESCRIPTION

The example embodiments discussed herein are directed to systems, apparatus, methods, and devices for SDHIP and EDHIP evaluation for a well drilled into a subterranean formation that has multiple formation layers. Drainage heights may be used to optimize well landing depth and well spacing, which also impacts well production performance. Drainage height estimation may be derived from oil, water, and/or other fluid production allocation and correlation results. SDHIP may provide an indication of hydrocarbons from rock volumes that are effectively extracted by hydraulic fracturing operations, as for example when the rock volume is fractured significantly. EDHIP may provide the same indication as SDHIP, but EHIP may also include rocks that are bypassed and are not effectively fractured. EDHIP may be related to well-to-well connectivity and/or fracture-driven interaction.

In some cases, use of example embodiments may allow for field operations that occur at the subsurface (e.g., in a fractured subterranean formation adjacent to a well) to be evaluated, which may lead to additional subterranean resources being extracted from the subsurface and/or increasing the injection capacity. Examples of such additional subterranean resources may include, but are not limited to, oil, water and natural gas. Use of example embodiments on production wells may be designed to comply with certain standards and/or requirements. Example embodiments may be used for wellbores drilled in conventional and/or unconventional (e.g., tight shale) subterranean formations and reservoirs. Equipment used for example embodiments of SDHIP and EDHIP evaluation for a well drilled into multiple subterranean formations (e.g., for production wells) may be located at a subsurface (e.g., within and adjacent to a wellbore in a subterranean formation) for production wells (e.g., wells undergoing a fracturing operation).

Example embodiments relate to an approach that analyzes various samples (e.g., core samples, cuttings, produced fluid samples, downhole fluid samples) to allocate production of oil and/or other subterranean resources among multiple wells and/or formations. SDHIP and EDHIP evaluation for a well drilled into multiple formation layers may include determining well placement and/or completion design (e.g., fracture geometry, landing depth, well spacing,) of a well (e.g., a new production well, an existing well). In addition, or in the alternative, SDHIP and EDHIP evaluation for a well drilled into multiple formation layers may be used for key decision making in unconventional (e.g., shale & tight asset development) formations. In addition, or in the alternative, SDHIP and EDHIP evaluation for a well drilled into multiple depths may be used to estimate original fracture in place (OFIP) and/or other factors, which may help optimize landing depth and well spacing, estimate the impact of special landing design (e.g., diagonal crossing, rolling cube), improve oil production, forecast oil related production issues, and improve reservoir modeling.

In addition, or in the alternative, SDHIP and EDHIP evaluation for a well drilled into multiple formation layers may be used to optimize reservoir simulation and production forecasts/optimization. In addition, or in the alternative, SDHIP and EDHIP evaluation for a well drilled into multiple formation layers may be used for production trouble shooting/root cause analysis for both parent and new wells.

SDHIP and EDHIP evaluation for a well drilled into multiple formation layers according to example embodiments may provide results and insights into one or more of a number of factors related to a well. Such factors may include but are not limited to oil geochemistry surveillance (e.g., for new and existing wells (defined below)), hydrocarbon properties, geoscience considerations (e.g., structural configurations, net pay, heterogeneity), engineering considerations (e.g., fluid properties, recovery mechanisms, fluid mobilities, fluid distribution, well productivity), and/or operational considerations (e.g., well types, completion, spacing, facility type and constraints, artificial lift, pattern type and spacing, injector/producer ratio).

As defined herein, improving well placement involves evaluating one or more aspects of a well so that the placement of that well is optimal for the purpose (e.g., production of subterranean resources, fluid compatibility) for which it and the adjacent wells are designed. Examples of such aspects may include, but are not limited to, horizontal distance between horizontal wellbores, vertical distance between vertical wellbores, and number of wellbores drilled and completed in a given volume of reservoir.

As defined herein, improving completion design involves evaluating one or more aspects of the completion design of a well so that such completion design is optimal for the purpose (e.g., production of subterranean resources, reduction of fluid compatibility issues) for which it and the adjacent wells are designed. Examples of such aspects may include, but are not limited to, the cluster spacing and orientation of perforations within each cluster.

As defined herein, a sample obtained from a well may be or include one or more of any of a number of materials. A sample obtained from a well may be or include a liquid, a solid, and/or a gas. In certain example embodiments, a sample obtained from a well includes some amount (e.g., trace amounts, 5% by volume or weight, 50% by volume or weight, 75% by volume or weight, 95% by volume or weight) of oil. Such oil may include one or more elements in addition to hydrogen and oxygen. In implementations, a sample may include core, cutting, oil based mud (OBM), base oil to make OBM, produced water, an excised portion of a subterranean formation (e.g., a core sample), and/or hydrocarbon (e.g., oil).

The use of the terms “about”, “approximately”, and similar terms applies to all numeric values, whether or not explicitly indicated. These terms generally refer to a range of numbers that one of ordinary skill in the art would consider as a reasonable amount of deviation to the recited numeric values (i.e., having the equivalent function or result). For example, this term may be construed as including a deviation of +10 percent of the given numeric value provided such a deviation does not alter the end function or result of the value. Therefore, a value of about 1% may be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein.

A “subterranean formation” refers to practically any volume under a surface. For example, it may be practically any volume under a terrestrial surface (e.g., a land surface), practically any volume under a seafloor, etc. A subterranean formation has one or more formation layers. Each subsurface volume of interest may have a variety of characteristics, such as petrophysical rock properties, reservoir fluid properties, reservoir conditions, hydrocarbon properties, or any combination thereof. For example, each subsurface volume of interest may be associated with one or more of: temperature, water composition, mineralogy, hydrocarbon type, hydrocarbon quantity, reservoir location, pressure, etc. Those of ordinary skill in the art will appreciate that the characteristics are many, including, but not limited to: shale gas, shale oil, tight gas, tight oil, tight carbonate, carbonate, vuggy carbonate, unconventional formation (e.g., a permeability of less than 25 millidarcy (mD) such as a permeability of from 0.000001 mD to 25 mD)), diatomite, mineral, etc.

In some embodiments, an unconventional formation may have a permeability of less than 25 millidarcy (mD) (e.g., 20 mD or less, 15 mD or less, 10 mD or less, 5 mD or less, 1 mD or less, 0.5 mD or less, 0.1 mD or less, 0.05 mD or less, 0.01 mD or less, 0.005 mD or less, 0.001 mD or less, 0.0005 mD or less, 0.0001 mD or less, 0.00005 mD or less, 0.00001 mD or less, 0.000005 mD or less, 0.000001 mD or less, or less). In some embodiments, the unconventional formation may have a permeability of at least 0.000001 mD (e.g., at least 0.000005 mD, at least 0.00001 mD, 0.00005 mD, at least 0.0001 mD, 0.0005 mD, 0.001 mD, at least 0.005 mD, at least 0.01 mD, at least 0.05 mD, at least 0.1 mD, at least 0.5 mD, at least 1 mD, at least 5 mD, at least 10 mD, at least 15 mD, or at least 20 mD).

An unconventional formation may include a permeability ranging from any of the minimum values described above to any of the maximum values described above. For example, in some embodiments, the unconventional formation may have a permeability of from 0.000001 mD to 25 mD (e.g., from 0.001 mD to 25 mD, from 0.001 mD to 10 mD, from 0.01 mD to 10 mD, from 0.1 mD to 10 mD, from 0.001 mD to 5 mD, from 0.01 mD to 5 mD, or from 0.1 mD to 5 mD).

The terms “formation”, “subterranean formation”, “subsurface formation”, “hydrocarbon-bearing formation”, “reservoir”, “subsurface reservoir”, “subsurface area of interest”, “subsurface region of interest”, “subsurface volume of interest”, and the like may be used synonymously. The term “subterranean formation” is not limited to any description or configuration described herein.

A “well” or a “wellbore” refers to a single hole, usually cylindrical when viewed in at least piecewise increments, that is drilled into a subsurface volume of interest. A well or a wellbore may be drilled in one or more directions. For example, a well or a wellbore may include a vertical well or section of the well, a horizontal well or section of the well, a deviated well or section of the well, and/or other type of well or section of the well. A well or a wellbore may be drilled in the subterranean formation for exploration and/or recovery of resources. A plurality of wells (e.g., tens to hundreds of wells) or a plurality of wellbores are often used in a field depending on the desired outcome.

A well or a wellbore may be drilled into a subsurface volume of interest using practically any drilling technique and equipment known in the art, such as geosteering, directional drilling, etc. Drilling the well may include using a tool, such as a drilling tool that includes a drill bit and a drill string. Drilling fluid, such as drilling mud, may be used while drilling in order to cool the drill tool and remove cuttings. Other tools may also be used while drilling or after drilling, such as measurement-while-drilling (MWD) tools, seismic-while-drilling tools, wireline tools, logging-while-drilling (LWD) tools, or other downhole tools. After drilling to a predetermined depth, the drill string and the drill bit may be removed, and then the casing, the tubing, and/or other equipment may be installed according to the design of the well. The equipment to be used in drilling the well may be dependent on the design of the well, the subterranean formation, the hydrocarbons and/or other subterranean resources being produced, and/or other factors.

A well may include a plurality of components, including but not limited to a casing, a liner, a tubing string, a sensor, a packer, a screen, a gravel pack, artificial lift equipment (e.g., an electric submersible pump (ESP)), and/or other components. If a well is drilled offshore, the well may include one or more of the previous components plus other offshore components, such as a riser. A well may also include equipment to control fluid flow into the well, control fluid flow out of the well, or any combination thereof. For example, a well may include a wellhead, a choke, a valve, and/or other control devices. These control devices may be located on the surface, in the subsurface (e.g., downhole in the well), or any combination thereof.

In some embodiments, the same control devices may be used to control fluid flow into and out of the well. In some embodiments, different control devices may be used to control fluid flow into and out of a well. In some embodiments, the rate of flow of fluids through the well may depend on the fluid handling capacities of the surface facility that is in fluidic communication with the well. The equipment to be used in controlling fluid flow into and out of a well may be dependent on the well, the subsurface region, the surface facility, and/or other factors. Moreover, sand control equipment and/or sand monitoring equipment may also be installed (e.g., downhole and/or on the surface). A well may also include any completion hardware that is not discussed separately. The term “well” may be used synonymously with the terms “borehole,” “wellbore,” or “well bore.” The term “well” is not limited to any description or configuration described herein.

“Hydraulic fracturing” is one way that hydrocarbons may be recovered (sometimes referred to as produced) from the formation. For example, hydraulic fracturing may entail preparing a fracturing fluid and injecting that fracturing fluid into the wellbore at a sufficient rate and pressure to open existing fractures and/or create fractures in the formation. The fractures permit hydrocarbons to flow more freely into the wellbore. In the hydraulic fracturing process, the fracturing fluid may be prepared on-site to include at least proppants. The proppants, such as sand or other particles, are meant to hold the fractures open so that hydrocarbons may more easily flow to the wellbore. The fracturing fluid and the proppants may be blended together using at least one blender. The fracturing fluid may also include other components in addition to the proppants.

The wellbore and the formation proximate to the wellbore are in fluid communication (e.g., via perforations), and the fracturing fluid with the proppants is injected into the wellbore through a wellhead of the wellbore using at least one pump (oftentimes called a fracturing pump). The fracturing fluid with the proppants is injected at a sufficient rate and pressure to open existing fractures and/or create fractures in the subsurface volume of interest. As fractures become sufficiently wide to allow proppants to flow into those fractures, proppants in the fracturing fluid are deposited in those fractures during injection of the fracturing fluid. After the hydraulic fracturing process is completed, the fracturing fluid is removed by flowing or pumping it back out of the wellbore so that the fracturing fluid does not block the flow of hydrocarbons to the wellbore. The hydrocarbons will typically enter the same wellbore from the formation and go up to the surface for further processing.

The equipment to be used in preparing and injecting the fracturing fluid may be dependent on the components of the fracturing fluid, the proppants, the wellbore, the formation, etc. However, for simplicity, the term “fracturing apparatus” is meant to represent any tank(s), mixer(s), blender(s), pump(s), manifold(s), line(s), valve(s), fluid(s), fracturing fluid component(s), proppants, and other equipment and non-equipment items related to preparing the fracturing fluid and injecting the fracturing fluid.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A.

In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C. In some embodiments, the item described by this phrase could include two or more components of type A (e.g., A1 and A2). In some embodiments, the item described by this phrase could include two or more components of type B (e.g., B1 and B2).

In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure may be inferred to that component. Conversely, if a component in a figure is labeled but is not described, the description for such component may be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of SDHIP and EDHIP evaluation for a well drilled into multiple formation layers will be described more fully hereinafter with reference to the accompanying drawings, in which example embodiments of SDHIP and EDHIP evaluation for a well drilled into multiple formation layers are shown. SDHIP and EDHIP evaluation for a well drilled into multiple formation layers may, however, be embodied in many different forms and should not be construed as limited to the example embodiments set forth herein. Rather, these example embodiments are provided so that this disclosure will be thorough and complete, and will fully convey the scope of SDHIP and EDHIP evaluation for a well drilled into multiple subterranean formations to those of ordinary skill in the art. Like, but not necessarily the same, elements (also sometimes called components) in the various figures are denoted by like reference numerals for consistency.

Terms such as “first”, “second”, “primary,” “secondary,” “above”, “below”, “inner”, “outer”, “distal”, “proximal”, “end”, “top”, “bottom”, “upper”, “lower”, “side”, “left”, “right”, “front”, “rear”, and “within”, when present, are used merely to distinguish one component (or part of a component or state of a component) from another. This list of terms is not exclusive. Such terms are not meant to denote a preference or a particular orientation, and they are not meant to limit embodiments of SDHIP and EDHIP evaluation for a well drilled into multiple formation layers. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

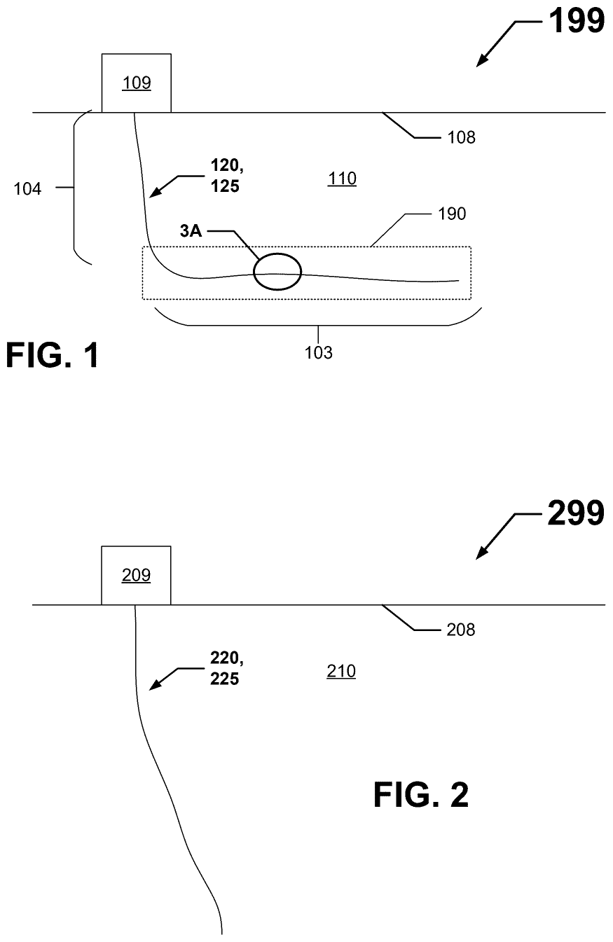

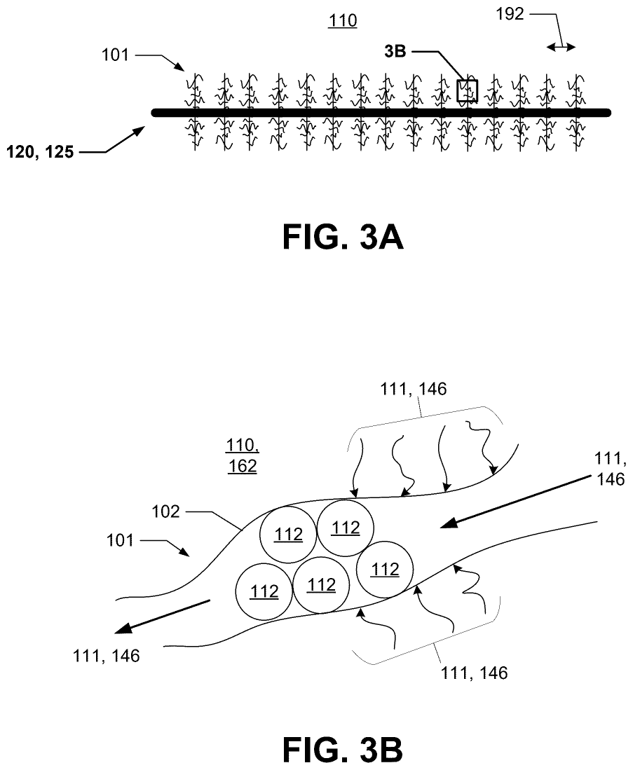

FIG. 1 shows a schematic diagram of a land-based field system 199 with which example embodiments may be used. FIG. 2 shows a schematic diagram of another land-based field system 299 with which example embodiments may be used. FIG. 3A shows a detail of a substantially horizontal section 103 of the wellbore 120 of FIG. 1. FIG. 3B shows a detail of a fracture 101 of FIG. 3A. The field system 199 of FIG. 1 includes a producing wellbore 120 disposed in a subterranean formation 110 using field equipment 109 (e.g., a derrick, a tool pusher, a clamp, a tong, drill pipe, casing pipe, a drill bit, a wireline tool, a fluid pumping system, a system for estimating a SDHIP for a well) located above a surface 108 and within the wellbore 120.

With respect to the system 199 of FIG. 1, once the wellbore 120 is drilled, a casing string 125 is inserted into the wellbore 120 to stabilize the wellbore 120 and allow for the extraction of subterranean resources (e.g., natural gas, oil, produced water) from the subterranean formation 110. Field equipment 109, located at the surface 108, is used to drill, encase, monitor, fracture, produce, and/or perform any other part of a field operation with respect to the wellbore 120. The wellbore 120 of FIG. 1 starts out with a substantially vertical section 104, and then has a substantially horizontal section 103. This configuration of the wellbore 120 is common for exploration and production of subterranean resources, such as oil and natural gas.

Similarly, with respect to the system 299 of FIG. 2, once the wellbore 220 is drilled, a casing string 225 is inserted into the wellbore 220 to stabilize the wellbore 220 from the subterranean formation 210. Field equipment 209, located at the surface 208, is used to drill, encase, monitor, fracture, produce, and/or perform any other part of a field operation with respect to the wellbore 220. The wellbore 220 of FIG. 2 is substantially vertical. This configuration of the wellbore 220 is common for injection wells.

Referring back to FIG. 1, the surface 108 may be ground level for an onshore application and the sea floor (or other similar floor under a body of water) for an offshore application. A body of oil may include, but it not limited to, trapped oil in rock, movable oil in rock, or free flowing oil in a reservoir. For offshore applications, at least some of the field equipment may be located on a platform that sits above the water level. The point where the wellbore 120 begins at the surface 108 may be called the wellhead.

While not shown in FIGS. 1 and 2, there may be multiple wellbores 120, 220, each with its own wellhead but that is located close to the other wellheads, drilled into the subterranean formation 110, 210 and having substantially vertical sections and/or horizontal sections 103 that are close to each other. In such a case, the multiple wellbores 120, 220 may be drilled at the same pad or at different pads. Example embodiments may be used to help determine, for example, the entry point of each wellbore (e.g., wellbore 120, wellbore 220) in the surface (e.g., surface 108, surface 208), the path of each wellbore in the subterranean formation (e.g., subterranean formation 110, subterranean formation 210), the depth of each wellbore, and the relative location of one wellbore to one or more of the other wellbores.

During the process of drilling the wellbore 120 of FIG. 1, as detailed in FIGS. 3A and 3B, core samples, cuttings, OBM, produced oil, formation oil, water 146 (e.g., produced water, formation water), and/or other subterranean resources 111 (e.g., relatively small amounts of oil or natural gas) may be extracted (or otherwise obtained) from downhole to the surface 108, where some of the field equipment 109 separates out at least some of the cuttings and recirculates the OBM back downhole. When the drilling process is complete, other operations, such as fracturing operations and production operations, may be performed. While the subterranean formation 110 may have naturally-occurring fractures 101 and some fractures 101 that may be created when drilling the wellbore 120, these fractures 101 may need to be enlarged and elongated, and additional fractures 101 may need to be created, in order to extract additional subterranean resources 111 (e.g., oil, natural gas) from the subsurface. The fractures 101 are shown to be located in the horizontal section 103 of the wellbore 120 in FIG. 1. The fractures 101, whether created and/or naturally occurring, may additionally or alternatively be located in other sections (e.g., a substantially vertical section 104, a transition area between a vertical section 104 and a horizontal section 103) of the wellbore 120. In some cases, a wellbore (e.g., wellbore 220) has no substantially horizontal sections, as shown in FIG. 2. Example embodiments may be used along any portion of a wellbore (e.g., wellbore 120, wellbore 220) where fractures 101 are located.

The subterranean formation 110 may include one or more of a number of formation types, including but not limited to shale, limestone, sandstone, clay, sand, and salt. In certain embodiments, a subterranean formation 110 may include one or more reservoirs in which one or more resources (e.g., oil, natural gas, water) may be located. One or more of a number of field operations (e.g., fracturing (e.g., hydraulic fracturing), coring, tripping, drilling, setting casing, extracting downhole resources, production) may be performed to reach an objective of a user with respect to the subterranean formation 110.

The wellbore 120 may have one or more of a number of segments or hole sections, where each segment or hole section may have one or more of a number of dimensions. Examples of such dimensions may include, but are not limited to, a size (e.g., diameter) of the wellbore 120, a curvature of the wellbore 120, a total vertical depth of the wellbore 120, a measured depth of the wellbore 120, and a horizontal displacement of the wellbore 120. There may be multiple overlapping casing strings of various sizes (e.g., length, outer diameter) contained within and between these segments or hole sections to ensure the integrity of the wellbore construction. In this case, one or more of the segments of the subterranean wellbore 120 is the substantially horizontal section 103.

As discussed above, inserted into and disposed within the wellbore 120 of FIGS. 1 and 2 are a number of casing pipes that are coupled to each other end-to-end to form the casing string 125 and the casing string 225, respectively. In these cases, each end of a casing pipe has mating threads (a type of coupling feature) disposed thereon, allowing a casing pipe to be directly or indirectly mechanically coupled to another casing pipe in an end-to-end configuration. The casing pipes of the casing string 125 and the casing string 225 may be indirectly mechanically coupled to each other using a coupling device, such as a coupling sleeve.

Each casing pipe of the casing string 125 and the casing string 225 may have a length and a width (e.g., outer diameter). The length of a casing pipe may vary. For example, a common length of a casing pipe is approximately 40 feet. The length of a casing pipe may be longer (e.g., 60 feet) or shorter (e.g., 10 feet) than 40 feet. The width of a casing pipe may also vary and may depend on the cross-sectional shape of the casing pipe. For example, when the shape of the casing pipe is cylindrical, the width may refer to an outer diameter, an inner diameter, or some other form of measurement of the casing pipe. Examples of a width in terms of an outer diameter may include, but are not limited to, 4½ inches, 7 inches, 7⅝ inches, 8⅝ inches, 10¾ inches, 13⅜ inches, and 14 inches.

The size (e.g., width, length) of the casing string 125 and the casing string 225 may be based on the information (e.g., diameter of the borehole drilled) gathered using field equipment with respect to the subterranean wellbore 120 and the subterranean wellbore 220, respectively. The walls of the casing string 125 and the casing string 225 have an inner surface that forms a cavity that traverses the length of the casing string 125 and the casing string 225. Each casing pipe may be made of one or more of a number of suitable materials, including but not limited to steel. Cement is poured into the wellbore 120 through the cavity and then forced upward between the outer surface of the casing string 125 and the wall of the subterranean wellbore 120. Similarly, cement is poured into the wellbore 220 through the cavity and then forced upward between the outer surface of the casing string 225 and the wall of the subterranean wellbore 220. In some cases, a liner may additionally be used with, or alternatively be used in place of, some or all of the casing pipes.

Referring to the system 199 of FIGS. 1, 3A, and 3B, once the cement dries, a number of fractures 101 are created in the subterranean formation 110 during a fracturing operation. The fractures 101 may be created in any of a number of ways known in the industry, including but not limited to hydraulic fracturing and/or other methods of generating fractures. The hydraulic fracturing process involves the injection of large quantities of fluids containing water, chemical additives, and proppant 112 into the subterranean formation 110 from the wellbore 120 to create fracture networks. A subterranean formation 110 naturally has fractures 101, but these naturally occurring fractures 101 have inconsistent characteristics (e.g., length, spacing) and so in some cases cannot be relied upon for extracting subterranean resources without having additional fractures 101, such as what is shown in FIG. 3A, created in the subterranean formation 110.

Operations that create fractures 101 in the subterranean formation 110 use any of a number of fluids that include proppant 112 (e.g., sand, ceramic pellets). When proppant 112 is used, some of the fractures 101 (also sometimes called principal or primary fractures) receive proppant 112, while a remainder of the fractures 101 (also sometimes called secondary fractures) do not have any proppant 112 in them.

As shown in FIG. 3B, the proppant 112 is designed to become lodged inside at least some of the created fractures 101 to keep those fractures 101 open after the fracturing operation is complete. The size of the proppant 112 is an important design consideration. Sizes (e.g., 40/70 mesh, 50/140 mesh) of the proppant 112 may vary. While the shape of the proppant 112 is shown as being uniformly spherical, and the size is substantially identical among the proppant 112, the actual sizes and shapes of the proppant 112 may vary. If the proppant 112 is too small, the proppant 112 will not be effective at keeping the fractures 101 open enough to effectively allow water 146 and/or other subterranean resources 111 to flow through the fractures 101 from the rock matrices 162 in the subterranean formation 110 to the wellbore 120. If the proppant 112 is too large, the proppant 112 may plug up the fractures 101, blocking the flow of the water 146 and/or other subterranean resources 111 through the fractures 101.

The use of proppant 112 in certain types of subterranean formation 110, such as shale, may be important. Shale formations typically have permeabilities on the order of microdarcys (μD) to nanodarcys (nD). When fractures 101 are created in such formations with low permeabilities, it is important to sustain the fractures 101 and their permeability and conductivity for an extended period of time in order to extract more of the subterranean resource 111. Example embodiments may also be applied to fluids used in other types of field operations, including but not limited to fracturing operations and injection wells.

Regardless of the type (e.g., conventional, unconventional) of subterranean formation 110, when proppant 112 and/or other similar components of a fracturing fluid are used in a fracturing operation, the proppant 112 and/or other similar components may be designed to become lodged within fractures 101 that result in principal fractures, which are designed to last (stay open) for a longer period of time as fluids (e.g., water 146, subterranean resources 111) flow therethrough. Fractures 101 that do not have proppant 112 and/or other similar components lodged therein may be referred to as secondary fractures, which may not last as long (close or reduce in size more quickly) as principal fractures.

The various created fractures 101 that originate at the wellbore 120 and extend outward into the rock matrices 162 in the subterranean formation 110 in this case have consistent penetration lengths perpendicular to the wellbore 120 and have consistent coverage along at least a portion of the lateral length (substantially horizontal section) of the wellbore 120. For example, created fractures 101 may be 50 meters high and 200 meters long. Further, the created fractures 101 may be spaced a distance 192 apart from each other. The distance 192 (e.g., 25 meters, 5 meters, 12 meters) may be optimized based on the permeability and the porosity of the rock matrix 162 of the subterranean formation 110.

The created fractures 101 create a volume 190 (also sometimes called a target volume 190) within the subterranean formation 110 where the rock matrix 162 of the subterranean formation 110 is connected to the high conductivity fractures 101 located a short distance away. In addition to different configurations of the fractures 101, other factors that may contribute to the viability of the subterranean formation 110 may include, but are not limited to, permeability of the rock matrix 162, capillary pressure, and the temperature and pressure of the subterranean formation 110. Each fracture 101, whether created or naturally occurring, is defined by a wall 102, also called a frac face 102 herein. The frac face 102 provides a transition between the paths formed by the rock matrices 162 in the subterranean formation 110 and the fracture 101. The subterranean resources 111 flow through the paths formed by the rock matrices 162 in the subterranean formation 110 into the fracture 101.

The rock matrices 162, as well as the rest of the subterranean formation 110, both without and outside the volume 190, have a certain amount of water 146 therein. The water 146 may be or include, for example, formation water from the formation matrix within the volume 190, moveable free formation water, and “external” water from non-targeted formation/sources (e.g., outside the target volume 190). These external sources of water 146 may include water from a nearby SWD source(s), a nearby hydrocarbon producing source, and/or other sources.

The water 146 may have any of a number of different components (e.g., minerals, chemical additives, acids, completion brine) in addition to formation water. The contents of water 146 in one part (e.g., outside the volume 190) of the subterranean formation 110 may be the same as, or different than, the contents of the water 146 in other parts (e.g., in the rock matrices 162) of the subterranean formation 110. In some cases, such as during a stage (e.g., a hydraulic fracturing stage) of a field operation, the fluids (e.g., fracturing fluid) used in that stage may mix with or include the water 146, thereby changing the contents or composition of the in situ water chemistry in parts (e.g., at or near the fractures 101) of the subterranean formation 110. The water 146 may include one or more of a number of types of water, including but not limited to sea water, brackish water, flowback or produced water, wastewater (e.g., reclaimed or recycled), brine (e.g., reservoir or synthetic brine), fresh water (e.g., fresh water comprises <1,000 ppm TDS), any other type of water, or any combination thereof.

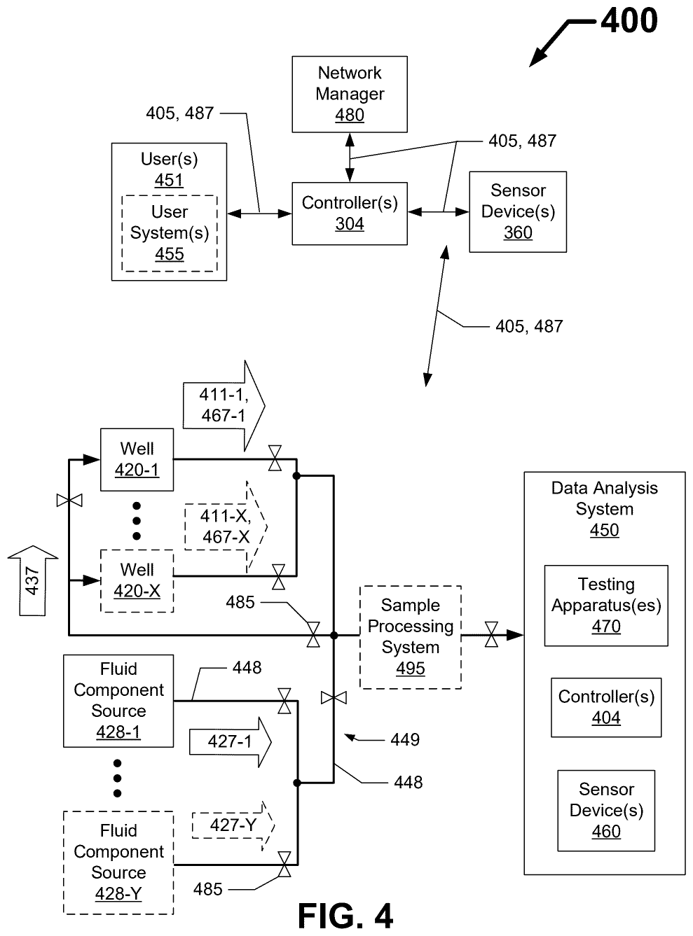

FIG. 4 shows a diagram of a system 400 for SDHIP and EDHIP evaluation for a well drilled into multiple formation layers according to certain example embodiments. The system 400 of FIG. 4 includes one or more solid and/or fluid component sources 428, one or more wells 420, an example data analysis system 450, an optional sample processing system 495, one or more controllers 304, one or more sensor devices 360, one or more users 451 (including one or more optional user systems 455), a network manager 480, a material conveyance system 448, and one or more valves 485. The data analysis system 450 in this case includes one or more testing apparatuses 470, one or more controllers 404, and one or more sensor devices 460.

The components shown in FIG. 4 are not exhaustive, and in some embodiments, one or more of the components shown in FIG. 4 may not be included in the example system 400. Any component of the system 400 may be discrete or combined with one or more other components of the system 400. Also, one or more components of the system 400 may have different configurations. For example, one or more sensor devices 360 may be disposed within or disposed on other components (e.g., the material conveyance system 448, a valve 485, a fluid component source 428, a well 420). As another example, a controller 304, rather than being a stand-alone device, may be part of one or more other components (e.g., a fluid component source 428, a well 420) of the system 400.

Referring to the description above with respect to FIGS. 1 through 3, the system 400 of FIG. 4 may include one or more wells 420 (in this case, well 420-1 through well 420-X). Each of the wells 420 of the system 400 may be substantially similar to the wells discussed above. Some or all of the wells 420 may be from a common pad. Each well 420 may produce oil, other subterranean resources 411, cuttings, other materials, or any combination thereof. In addition, or in the alternative, one or more core samples may be taken from one or more of the wells 420. From these materials (e.g., core samples, cuttings, produced fluids) that flow uphole from each well 420 to the surface, one or more samples 467 may be obtained. Each sample 467 that is obtained may be transported through the material conveyance system 448, through the optional sample processing system 495, and to the data analysis system 450. Over time, a well 420 may be used for different purposes. For example, well 420-1 may be used as a development well at one time and as a production well at another time.

A sample 467 of FIG. 4 may include a liquid, a solid, and/or a gas. For example, a sample 467 (also sometimes called an endmember may be or include oil, which may be substantially the same as the oil discussed above. Specifically, the oil may be any type of oil, including but not limited to the produced oil, extracted oil, downhole oil sample, OBM oil, and/or any other type of oil. Each sample 467 is specifically categorized as being from a particular well 420 and originate from a particular depth or range of depths from within the well 420. For example, samples 467-1 are from well 420-1, and samples 467-X are from well 420-X. A sample 467 may be or include produced oil, downhole fluids, produced fluids, cuttings, core samples, formation water, fracturing fluid (fracturing water), and/or hydrocarbon (e.g., natural gas).

One example of a sample 467 may be in the form of a core sample. In such a case, the oil extracted from the core sample is from a single formation layer, and so is not mixed with oil from other formation layers for the well 420. Also, a sample 467 in the form of a core sample may provide good resolution on drainage height (e.g., SDHIP, EDHIP) when core samples are collected in regular intervals (e.g., every 20 feet to 50 feet) in the well. However, samples 467 in the form of core samples, when used as the sole source of measurements of parameters, have a number of drawbacks. For example, core samples taken from heterogeneous reservoirs (or formation layers) may have large uncertainty. As another example, core samples may not address the free flowing oil (movable oil) in a reservoir. As another example, extracted oil from core samples does not represent produced oil.

As yet another example, measurements from core samples may present uncertainty because the environment (e.g., temperature, pressure, water, contact surface area, wettability, etc.) under which oil is extracted is different from the environment under which oil is produced. As still another example, core samples are costly (even more so when a wax coating is needed), difficult to obtain, and pause further progress of a field operation of a well. As yet another example, differences on a whole oil gas chromatogram between extracted oil and produced oil, especially for unconventional reservoirs, may lead to uncertainty on data analysis and interpretation.

Another example of a sample 467 may be in the form of cuttings. In such a case, cuttings are relatively easy to collect, but they require processing to obtain useful measurements of parameters. Also, cuttings may be collected in known intervals within the well 420 and can provide useful information on lateral heterogeneity from heel to toe sections. However, samples 467 in the form of cuttings, when used as the sole source of measurements of parameters, have a number of drawbacks. For example, knowing a specific depth of cuttings may be difficult because of turbulence and other factors that may cause a delay in the cuttings getting to the surface (e.g., surface 108).

As another example, measurements from cuttings may present uncertainty because the environment (e.g., temperature, pressure, water, contact surface area, wettability, etc.) under which oil is extracted is different from the environment under which oil is produced. As yet another example, differences on a whole oil gas chromatogram between extracted oil and produced oil, especially for unconventional reservoirs, may lead to uncertainty on data analysis and interpretation. As still another example, the presence of OBM in a sample 467 may adversely affect data analysis and interpretation, which is problematic because OBM is commonly used in drilling fluid.

Another example of a sample 467 may be in the form of produced oil. In such a case, samples 467 in the form of produced oil may represent overall produced oil from a well 420, even for wells 420 with complicated geology conditions. Also, produced oil may capture movable oil signatures and other types of actual field conditions. Further, samples 467 of produced oil may be relatively easy to obtain and may require little or no processing. However, samples 467 in the form of produced oil, when used as the sole source of measurements of parameters, have a number of drawbacks. For example, produced oil may contain oil signatures from other formation layers that are above and/or below the target formation layer. As another example, it may be difficult to allocate produced oil to a specific depth within a well 420.

The samples 467 are moved from each well 420 toward the data analysis system 450 using a conveyance system 448. The conveyance system 448 may be configured to extract the samples 467 from a well 420 and convey the samples 467 through the conveyance system 448 toward the data analysis system 450. The conveyance system 448 may additionally or alternatively be configured to extract a fluid component 427 from a fluid component source 428 and convey the fluid component 427 through the conveyance system 448 to the data analysis system 450,

As discussed above, the system 400 may also include one or more fluid component sources 428. Each fluid component source 428 may hold one or more fluid components 427. A fluid component source 428 may include, but is not limited to, a natural vessel (e.g., land that forms walls to contain a liquid, a subterranean cavity that holds carbon dioxide or other gas or liquid) and a man-made storage tank or other type of vessel. Each fluid component 427 may be, include, or be in the form of a liquid, a solid, and/or a gas. A single fluid component 427 or a mixture of multiple fluid component 427 may be disposed in a fluid component source 428.

Examples of a fluid component 427 may include, but are not limited to, carbon dioxide, gas with various concentrations of CO₂ (e.g., in liquid form, in gas form, in produced gas from a field operation, from a source external to a field operation), hydrocarbons, a chemical used for a fracturing operation, water that does not come from a well 420, methane, H₂S, nucleation catalyzing metals, an alkali salt (e.g., NaOH), sodium bicarbonate (NaHCO₃), sodium carbonate (Na₂CO₃), polymers and/or other substances, and flocculation agents. In some cases, multiple fluid components 427 may be combined to form a fluid 437. In some other cases, a single fluid component 427 may be a fluid 437.

A fluid component 427 may serve one or more purposes in one or more field operations. For example, a fluid component 427 may be used in a fluid 437 to generate and/or enhance fractures 101 in a subterranean formation 110 adjacent to a well 420 during a fracturing operation. As another example, a fluid component 427 may be carbon dioxide, a gas stream containing carbon dioxide (e.g., stored, produced), or any combination thereof, which may be used during injection of an injection well. One of ordinary skill in the art will appreciate that other fluid components 427 and/or combinations thereof are possible in example embodiments. A fluid 437 may be or include a liquid, a solid, and/or a gas.

The conveyance system 448 may include one or more of a number of pieces of equipment to perform its function. Examples of such equipment may include, but are not limited to, a compressor, a motor, a pump, a conveyer, a truck or other vehicle, a rail system, a crane, a shaker, a vibrator, piping, a valve (e.g., valve 485), a controller (e.g., controller 404), and a sensor device (e.g., sensor device 460). Some or all of the conveyance system 448 may operate using a controller (e.g., controller 404). In addition, or in the alternative, one or more users 451 may perform one or more of the various functions required to move some or all of the samples 467, one or more of the fluid components 427, some or all of the subterranean resources, and/or one or more of the fluids 437 using the conveyance system 448. The conveyance system 448 (including the collection area 449) may include any components, devices, subsystems, etc. that transport the samples 467, the subterranean resources 411, the fluid components 427, and the fluid 437 within the system 400 from one component to another component. The conveyance system 448 may be configured to transport solids, liquids, and/or gases.

For example, in order to transport liquids and gases within the system 400, the conveyance system 448 may include piping. In such a case, the piping may include multiple pipes, ducts, elbows, joints, sleeves, collars, and similar components that are coupled to each other (e.g., using coupling features such as mating threads) to establish a network for transporting such liquids and/or gases within the system 400. Each component of the piping may have an appropriate size (e.g., inner diameter, outer diameter) and be made of an appropriate material (e.g., steel, PVC, copper) to safely and efficiently handle the pressure, temperature, flow rate, and other characteristics of the liquids and/or gases that flow therethrough. As another example, in order to transport solids within the system 400, the conveyance system 448 may include conveyer belts, trucks, bulldozers, backhoes, and/or other similar equipment.

There may be a number of valves 485 placed in-line with the conveyance system 448 (or portions thereof) at various locations in the system 400 to control the flow of the samples 467, the fluid components 427, the subterranean resources 411, and/or the fluid 437 in liquid and/or gas form. A valve 485 may have one or more of any of a number of configurations, including but not limited to a guillotine valve, a ball valve, a gate valve, a butterfly valve, a pinch valve, a needle valve, a plug valve, a diaphragm valve, and a globe valve. One valve 485 may be configured the same as or differently compared to another valve 485 in the system 400. Also, one valve 485 may be controlled (e.g., manually by a user 451, automatically by a controller 404 of the data analysis system 450) the same as or differently compared to another valve 485 in the system 400.

In some cases, positioned within the material conveyance system 448 between the wells 420, the fluid component sources 428, and the data analysis system 450 may be an optional sample processing system 495. Such a sample processing system 495 may be or include part of the field equipment 109 discussed above. The sample processing system 495 may be designed to separate cuttings, other subterranean resources 411 (e.g., oil, natural gas), and/or other elements from the samples 467 as the samples 467 are prepared for testing in the data analysis system 450 and/or for recirculation into a well 420 (e.g., the same well 420 from which the samples 467 are obtained, another well 420 (e.g., a SWD well)).

Such a sample processing system 495 may include one or more of a number of various pieces of equipment. Such equipment may include, but is not limited to, a pump, a motor, a filter, a centrifuge, a heater, a blower, a condenser, a vessel, a funnel, a strainer, a separator, an agitator, a paddle, a circulating system, an aerator, a heat exchanger, a column, a test tube, a separator, a mixer (e.g., a centrifuge mixer, a desander, a tumbler mixer, a homogenizer, a static mixer, a drum mixer, a fluidization mixer, agitator mixers, paddle mixers, an emulsifier, a drum mixer, a pail mixer, a convective mixer, an agitator, a batch mixer, and a ribbon mixer), a controller (e.g., controller 304, controller 404), and a sensor device (e.g., sensor device 360, sensor device 460).

The sample processing system 495 may operate substantially continuously (e.g., as when the samples 467 and/or the fluid components 427 substantially continuously flow into the sample processing system 495) or at intervals (e.g., as when the samples 467 and/or the fluid components 427 are introduced into the sample processing system 495 intermittently). The sample processing system 495 may be or include a single apparatus (with or without multiple portions) or multiple apparatuses (or multiple portions thereof) that operate in series and/or in parallel with each other. As an example, the sample processing system 495 may include a temperature conditioning portion, a mixing portion, a drying portion, and a separating portion that operate in series with each other. As another example, the sample processing system 495 may include multiple mixers that operate in parallel with each other, where each mixer may mix one or more fluid components 427 and/or one or more of the samples 467 into a different fluid 437 simultaneously. Some or all of the sample processing system 495 may be controlled by one or more controllers 404 of the data analysis system 450.

The sample processing system 495 may control various aspects (e.g., temperature, pressure, flow rate, moisture/dryness) of the samples 467, the fluid components 427, the subterranean resources 411, and/or the fluid 437. In some cases, the sample processing system 495 is designed to subject the samples 467, the fluid components 427, the subterranean resources 411, and/or the fluid 437 to conditions (e.g., pressure, temperature, flow rate) that simulate the conditions at the subsurface (e.g., corresponding downhole conditions of the fractures 101 and rock matrix in the subterranean formation 110 adjacent to the wellbore 120).

In some cases, some or all of the sample processing system 495 may be operated, paused, and/or stopped so that the samples 467, the fluid components 427, the subterranean resources 411, and/or the fluid 437 may be evaluated by the testing apparatus 470. Testing by the testing apparatus 470 may be controlled by a user 451 (e.g., a human being) and/or a controller 404 of the data analysis system 450. Testing by the testing apparatus 470 may be based on historical data and/or field data (e.g., measurements from sensor devices 460). Testing by the testing apparatus 470 may generate test scenarios or expected results. Testing by the testing apparatus 470 may include the use of one or more algorithms 533, one or more protocols 532, and/or stored data 534 (all discussed below).

Whether inside the sample processing system 495 or in a collection area 449 (e.g., a header, a manifold), some or all of the samples 467 and/or some or all of the fluid components 427 may be introduced to each other. Conditions (e.g., temperature, pressure) in some or all of the sample processing system 495 may vary and may be customized or otherwise controlled (e.g., to represent field operating conditions).

To control the composition of a fluid 437 at a given point in time, the amount of one or more of the samples 467 and/or the amount of the one or more fluid components 427 that are released or withdrawn from the one or more wells 420 and/or the one or more fluid component sources 428, respectively, may be regulated in real time. This regulation may be performed automatically by a controller (e.g., controller 404) and/or manually by a user 451 (which may include an associated user system 455). This regulation may be performed using equipment such as the sample processing system 495 (including portions thereof), pumps, compressors, field equipment (e.g., field equipment 109), the conveyance system 448, valves 485, regulators, sensor devices 460, etc. The samples 467 of a well 420 and a fluid component 427 of a fluid component source 428 may have any of a number of different compositions that are naturally occurring or man-made.

The data analysis system 450 of the system 400 may be configured to perform an analysis on one or more of the samples 467 from one or more of the wells 420 for the purpose of estimating a SDHIP for a well drilled into multiple subterranean formations. In addition, the data analysis system 450 of the system 400 may be configured to perform a chemical and/or other type of analysis of one or more of the subterranean resources 411 from one or more of the wells 420, one or more of the fluid components 427 from one or more of the fluid component sources 428, and/or one or more of the fluids 437 that are delivered to one or more of the wells 420. As a result, the system 400 (and more specifically the data analysis system 450) may be used to estimate a SDHIP for a well drilled into multiple subterranean formations using an analysis of the samples 467, an analysis of the subterranean resources 411, an analysis of the fluid components 427, and/or an analysis of the fluids 437. As a result, example embodiments may be used, for example, to optimize landing depth and well spacing, estimate the impact of special landing design (e.g., diagonal crossing and rolling cube), improve oil production, forecast oil related production issues, and improve reservoir modelling.

As discussed above, the data analysis system 450 may include one or more components. For example, in this case, the data analysis system 450 includes one or more testing apparatuses 470, one or more controllers 404, and one or more sensor devices 460. Each testing apparatus 470 may be configured to test one or more of the samples 467, one or more of the subterranean resources 411, one or more of the fluid components 427, and/or one or more of the fluids 437. A single testing apparatus 470 may perform multiple tests (e.g., on a single fluid component 427, on multiple samples 467 from a single well 420, on a fluid 437 and on a subterranean resource 411) simultaneously.

When the data analysis system 450 has multiple testing apparatuses 470, one testing apparatus 470 may operate in conjunction with, or independently of, one or more of the other testing apparatuses 470. Further, when the data analysis system 450 has multiple testing apparatuses 470, one testing apparatus 470 may be configured (e.g., in terms of equipment, in terms of operating capability, in terms of control) the same as, or differently than, one or more of the other testing apparatuses 470. The operation of a testing apparatus 470 may be controlled by a user 451 (including an associated user system 455) and/or a controller 404 of the data analysis system 450.

A testing apparatus 470 may include or interact with one or more sensor devices 460 (discussed below) to perform one or more of its functions. Testing performed by a testing apparatus 470 may use or include historical data and/or field data (e.g., measurements from sensor devices 460). Testing may generate test scenarios or expected results. Testing may include the use of process chemistry simulators, fluid electrolyte modeling, chemistry calculations using field/historical data to model the process, etc.

A controller 404 of the data analysis system 450 may be configured to evaluate, using results of tests performed by a testing apparatus 470, the samples 467 obtained from one or more wells 420, one or more of the subterranean resources 411 obtained from one or more of the wells 420, one or more of the fluid components 427 obtained from one or more of the fluid component sources 428, and/or one or more of the fluids 437 that are delivered to one or more of the wells 420. In some cases, a controller 404 may be configured to facilitate the implementation of a test on a sample 467, control the parameters of a test performed on a sample 467, and/or take other actions associated with the testing of samples 467.

In some cases, one or more controllers 404 of the data analysis system 450 may be used to control or facilitate control of the implementation of the output of any of the algorithms 533 (e.g., models) of the data analysis system 450. For example, when the output of an algorithm 533 indicates a particular amount and type of chemical component or compound to add to a fluid 437 for use in one or more wells 420 during a current or planned field operation, one or more of the controllers 404 of the data analysis system 450 may obtain the amount and type of chemical component or compound from one or more of the fluid component sources 428, mix the chemical component or compound into a fluid 437 using the sample processing system 495, and deliver the resulting fluid 437 to one or more of the wells 420 using the conveyance system 448.

As another example, based on the output of an algorithm 533, a controller 404 of the data analysis system 450 may determine that a new well 420 would be beneficial to the overall production of subterranean resources 411 from the network of wells 420. In such a case, one or more of the controllers 404 of the data analysis system 450 may generate a drilling plan for the new well 420, implement or facilitate implementation of the drilling plan, evaluate the drilling plan in real time as the new well is drilled, make any revisions to the drilling plan based on the real-time evaluation, and implement or facilitate implementation of the revisions to the drilling plan.

A testing apparatus 470 of the data analysis system 450 may be configured to process one or more solids in addition to fluids. In such a case, the testing apparatus 470 may be configured to provide analysis of one or more of the samples 467, including but not limited to Fourier transformed infrared spectroscopy (FT-IR), x-ray fluorescence (XRF), gas chromatography, gas chromatograph-mass spectrometry, two dimensional gas chromatography (with FID and/or MS), three dimensional gas chromatography (with FID and/or MS), SARA analysis, biomarker analysis, stable carbon isotope analysis, stable sulfur isotope analysis, total sulfur analysis, elemental analysis, DNA sequencing for oil, UV-Vis spectroscopy, transmission spectroscopy, etc. The testing apparatus 470 may include one or more of any of a number of different equipment, including but not limited to a sifter, a shaker, a screen, a motor, a controller (e.g., controller 404), and a sensor device (e.g., sensor device 460). In some cases, the testing apparatus 470, or portions thereof, may operate using a controller 404. In addition, or in the alternative, one or more users 451 may perform one or more of the various functions required to operate some or all of the testing apparatus 470.

Each testing apparatus 470 of the data analysis system 450 may be configured to test the samples 467 of one or more wells 420, the subterranean resources 411 of one or more wells 420, one or more fluid components 427, and one or more of the fluids 437. A testing apparatus 470 may be used in conjunction with one or more sensor devices 460 of the data analysis system 450. A testing apparatus 470 may be or include a vessel (e.g., a bottle, a column, a test tube) inside of which various materials (e.g., samples 467 from a well 420, a fluid component 427, a fluid 437) are disposed for testing. In some cases, the materials placed in a testing apparatus 470 are first processed in the sample processing system 495. In any case, the materials are provided to a testing apparatus 470 by the conveyance system 448. A testing apparatus 470 may be used to test samples 467, a fluid component 427, a subterranean resource 411, a fluid 437, and/or any other component during a fracturing operation of one or more wells 420, during shut-in of a well 420, during pre-production of a well 420, during production of a well 420, and/or at any other time.

A testing apparatus 470 may include one or more components or pieces of equipment to perform its function. Examples of such components or pieces of equipment may include, but are not limited to, a membrane, a sifter, a shaker, a screen, an immersion separator, a reverse osmosis membrane, a nanofiltration membrane, a pH adjustment apparatus, a softening apparatus, a motor, a controller (e.g., controller 404), and a sensor device (e.g., sensor device 460). In some cases, a testing apparatus 470, or portions thereof, may operate using a controller 404. In addition, or in the alternative, one or more users 451 (e.g., a human being) may perform some or all of the various functions required to operate some or all of a testing apparatus 470.

The data analysis system 450 may include one or more sensor devices 460. Each sensor device 460 includes one or more sensors that measure one or more parameters (e.g., pressure, flow rate, temperature, humidity, fluid content, voltage, current, permeability, porosity, rock characteristics, chemical elements in a fluid, chemical elements in a solid, concentrations, etc.). Examples of a sensor of a sensor device 460 may include, but are not limited to, a temperature sensor, a flow sensor, a pressure sensor, a gas spectrometer, a voltmeter, an ammeter, a permeability meter, a spectrograph, a gas chromatograph a porosimeter, and a camera. A sensor device 460 may be a stand-alone device or integrated with another component of the data analysis system 450.

A parameter measured by a sensor device 460 may be associated with one or more components of the system 400. For example, a sensor device 460 may be configured to measure a parameter (e.g., flow rate, pressure, temperature, composition, concentration) of one or more samples 467, of a fluid component 427, of a subterranean resource 411, and/or of a fluid 437 at any location (e.g., between a well 420 and the data analysis system 450, between a fluid component source 428 and the data analysis system 450, within the sample processing system 495, etc.) of the system 400 at a particular time.

As another example, a sensor device 460 may be configured to determine how open or closed a valve 485 within the system 400 is. As yet another example, one or more sensor devices 460 may be used to identify the water chemistry and/or other characteristics of a sample 467. As still another example, one or more sensor devices 460 may be used to identify the contents of a fluid 437. As yet another example, one or more sensor devices 460 may be used to identify the contents of a fluid component 427.

In some cases, the measurements made by a number of sensor devices 460, each measuring a different parameter, may be used in isolation or in combination to determine and/or confirm whether a controller 404 should take a particular action (e.g., operate a valve 485, operate or adjust the operation of a testing apparatus 470, operate or adjust the operation of the sample processing system 495). When a sensor device 460 includes its own controller 404 (or portions thereof), then the sensor device 460 may be considered a type of computer device, as discussed below with respect to FIG. 6.

The data analysis system 450 may include one or more controllers 404. A controller 404 of the data analysis system 450 communicates with and in some cases controls one or more of the other components (e.g., a sensor device 460, a testing apparatus 470) of the data analysis system 450 and/or one or more other components (e.g., a sensor device 360, a controller 304, the conveyance system 448, one or more valves 485, a fluid component source 428, the sample processing system 495) of a remainder of the system 400. A controller 404 performs any of a number of functions that include, but are not limited to, obtaining and sending data, evaluating data, following protocols, running algorithms, and sending commands.

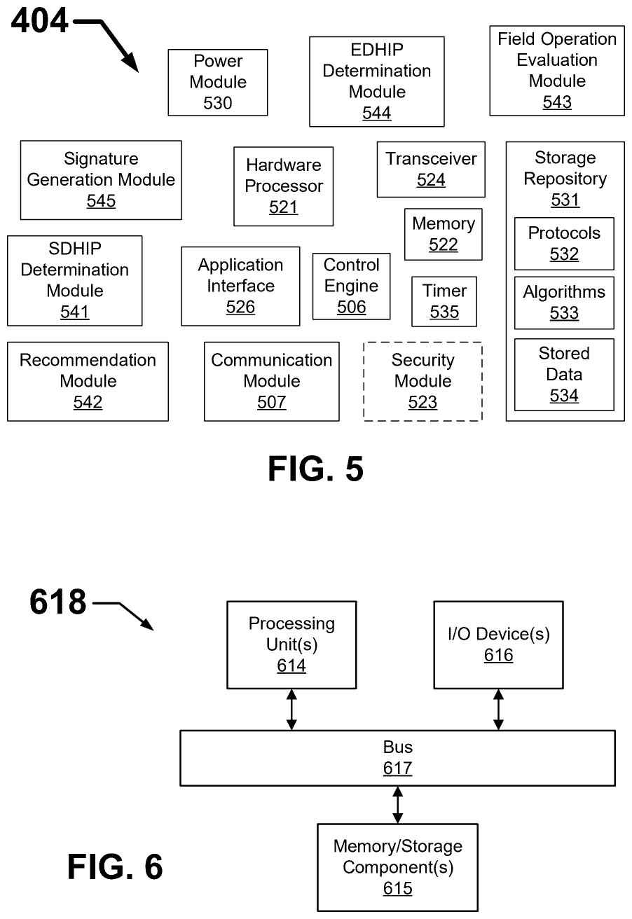

A controller 404 may include one or more of a number of components. For example, as shown in FIG. 5, such components of a controller 404 may include, but are not limited to, a control engine 506, a SDHIP determination module 541, a LDHIP determination module 544, a signature determination module 544, a recommendation module 542, a field operation evaluation module 543, a communication module 507, a timer 535, a power module 530, a storage repository 531, a hardware processor 521, a memory 522, a transceiver 524, an application interface 526, and, optionally, a security module 523. A controller 404 (or components thereof) may be located at or near the various components of the data analysis system 450. In addition, or in the alternative, the controller 404 (or components thereof) may be located remotely from (e.g., in the cloud, at an office building) the various components of the data analysis system 450.

In certain example embodiments, a controller 404 may be used to implement and/or alter a parameter (e.g., a design parameter for a current wellbore 420, an operating parameter for a current wellbore 420, a design parameter for a different wellbore 420 in the subterranean formation (e.g., subterranean formation 110, subterranean formation 210) an operating parameter for a different wellbore 420 in the subterranean formation) of a field operation. When there are multiple controllers 404 (e.g., one controller 404 for a testing apparatus 470, another controller 404 for a fluid component source 428, yet another controller 404 for the sample processing system 495), each controller 404 may operate independently of each other. Alternatively, two or more of the multiple controllers 404 may work cooperatively with each other. As yet another alternative, one of the controllers 404 may control some or all of one or more other controllers 404 in the system 400 or portion thereof. Each controller 404 may be considered a type of computer device, as discussed below with respect to FIG. 6.

The storage repository 531 may be a persistent storage device (or set of devices) that stores software and data used to assist a controller 404 in communicating with one or more other components of a system, such as the users 451 (including associated user systems 455), each well 420, each fluid component source 428, the sample processing system 495, the controllers 304, the sensor devices 360, other controllers 404 of the data analysis system 450, the network manager 480, the sensor devices 460, etc. of the system 400 of FIG. 4 above. In one or more example embodiments, the storage repository 531 stores one or more protocols 532, one or more algorithms 533, and stored data 534.

The protocols 532 of the storage repository 531 may be any procedures (e.g., a series of method steps) and/or other similar operational processes that the control engine 506 of the controller 404 follows based on certain conditions at a point in time. The protocols 532 may include any of a number of communication protocols that are used to send and/or obtain data between a controller 404 and other components of a system (e.g., the system 400). Such protocols 532 used for communication may be time-synchronized protocols. Examples of such time-synchronized protocols may include, but are not limited to, a highway addressable remote transducer (HART) protocol, a WirelessHART protocol, and an International Society of Automation (ISA) 100 protocol. In this way, one or more of the protocols 532 may provide a layer of security to the data transferred within a system (e.g., the system 400). Other protocols 532 used for communication may be associated with the use of Wi-Fi, Zigbee, visible light communication (VLC), cellular networking, BLE, UWB, and Bluetooth.

The algorithms 533 may be any formulas, mathematical models, forecasts, simulations, and/or other similar tools that the control engine 506 of a controller 404 uses to reach a computational conclusion. For example, one or more algorithms 533 may be used, in conjunction with one or more protocols 532, to assist a controller 404 to determine when to start, adjust, and/or stop the operation of a well 420, a fluid component source 428, a testing apparatus 470, the sample processing system 495, a sensor device 460, and/or another controller 404 of the data analysis system 450. As another example, one or more algorithms 533 may be used, in conjunction with one or more protocols 532, to assist a controller 404 to determine when to have a sensor device 460 measure a parameter and subsequently assist the controller 404 in performing a calculation or make a determination using the measurement.

As yet another example, one or more algorithms 533 may be used, in conjunction with one or more protocols 532, to assist a controller 404 to identify an optimal scenario (e.g., most cost effective, most effective landing depth, most effective well spacing, most effective special landing design, optimal oil production forecast, most effective forecasting of production issues, optimal improvements to one or more algorithms 533 (e.g., models)) of the well 420 and/or adjacent wells based on the analysis of the samples 467. As still another example, one or more algorithms 533 may be used, in conjunction with one or more protocols 532, to assist a controller 404 in identifying and tracking the origin (e.g., in terms of a particular well 420) of the various samples 467 that are extracted from a particular well 420 and analyzed using the data analysis system 450.

Stored data 534 may be any data associated with a field (e.g., the subterranean formation 110, the fractures 101 within the volume 190 adjacent to a wellbore 120, the characteristics of proppant 112 used in a field operation, measured parameters and analysis of the samples 467), other fields (e.g., other wellbores and subterranean formations), the other components (e.g., the user systems 455, the testing apparatuses 470, the sensor devices 460, the sensor devices 360, the controllers 304, the fluid components 427, the sample processing system 495), including associated equipment (e.g., motors, pumps, compressors), of the system 400, measurements made by the sensor devices 460 and the sensor devices 360, threshold values, tables, results of previously run or calculated algorithms 533, updates to protocols 532, user preferences, and/or any other suitable data. Such data may be any type of data, including but not limited to historical data, present data, and future data (e.g., forecasts). The stored data 534 may be associated with some measurement of time derived, for example, from the timer 535.

Examples of a storage repository 531 may include, but are not limited to, a database (or a number of databases), a file system, cloud-based storage, a hard drive, flash memory, some other form of solid-state data storage, or any suitable combination thereof. The storage repository 531 may be located on multiple physical machines, each storing all or a portion of the communication protocols 532, the algorithms 533, and/or the stored data 534 according to some example embodiments. Each storage unit or device may be physically located in the same or in a different geographic location.

The storage repository 531 may be operatively connected to the control engine 506. In one or more example embodiments, the control engine 506 includes functionality to communicate with the users 451 (including associated user systems 455), the testing apparatuses 470, the sample processing system 495, the sensor devices 460, the sensor devices 360, the controllers 304, the network manager 480, and/or the other components in the system 400. More specifically, the control engine 506 sends information to and/or obtains information from the storage repository 531 in order to communicate with the users 451 (including associated user systems 455), the testing apparatuses 470, the sample processing system 495, the sensor devices 460, the sensor devices 360, the controllers 304, the network manager 480, and/or the other components of the system 400. As discussed below, the storage repository 531 may also be operatively connected to the communication module 507 in certain example embodiments.

In certain example embodiments, the control engine 506 of a controller 404 controls the operation of one or more components (e.g., the communication module 507, the timer 535, the transceiver 524) of the controller 404. For example, the control engine 506 may activate the communication module 507 when the communication module 507 is in “sleep” mode and when the communication module 507 is needed to send data obtained from another component (e.g., a sensor device 460) in the system 400. In addition, the control engine 506 of a controller 404 may control the operation of one or more other components (e.g., a testing apparatus 470, the sample processing system 495, a fluid component source 428, a well 420), or portions thereof, of the system 400.

The control engine 506 of a controller 404 may communicate with one or more other components of the system 400. For example, the control engine 506 may use one or more protocols 532 to facilitate communication with the sensor devices 460 to obtain data (e.g., measurements of various parameters, such as water chemistry, intensity, temperature, pressure, and flow rate), whether in real time or on a periodic basis and/or to instruct a sensor device 460 to take a measurement. As another example, the control engine 506 may use one or more algorithms 533 and/or protocols 532 to decide which testing methods (e.g., whole oil gas chromatograph, oil biomarker GC-MS analysis, stable carbon isotope, stable sulfur isotope, SARA, sulfur analysis, Ni/V analysis, DNA sequencing for oil samples, water analysis, alkylbenzene analysis, 2D/3D GC-MS for detailed oil component analysis) to perform on the samples 467 from a well 420.

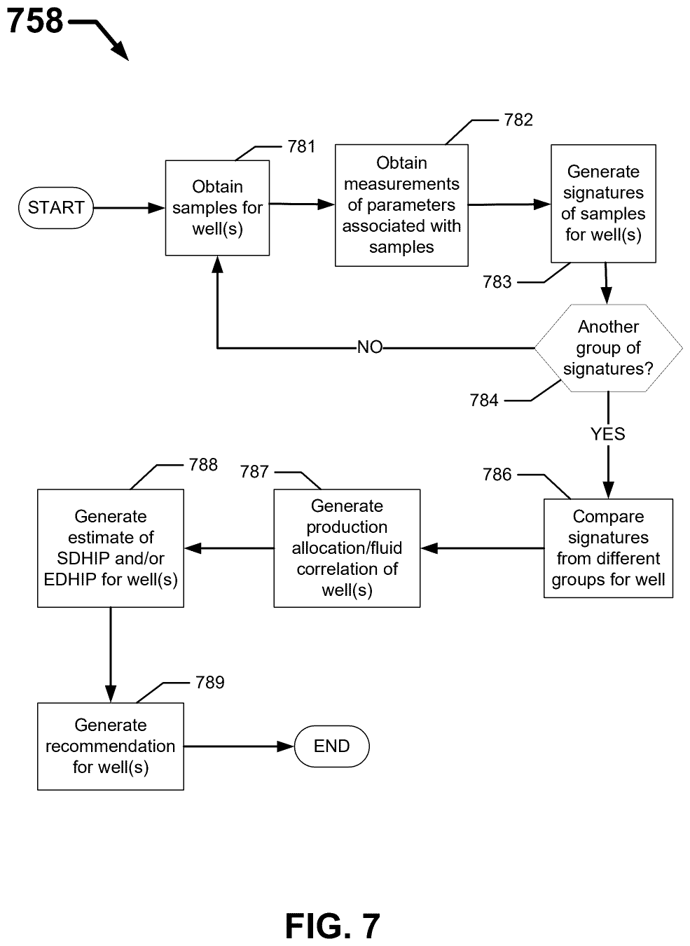

As yet another example, the control engine 506 may use one or more algorithms 533 and/or protocols 532 to generate a new algorithm 533 that provides expected results using the historical analysis of samples 467 for a well 420. As still another example, the control engine 506 may use one or more algorithms 533 and/or protocols 532 to determine, using the results of testing the one or more parameters associated with the samples 467, a volume of the field operation fluid (e.g., fracturing fluid when the field operation includes fracturing) when the field operation causes the migration of fluids from the area proximate to one well 420 to an adjacent area proximate to an adjacent well 420, whether during and/or after the field operation. A number of other capabilities of the control engine 506 (as well as the controller 404 as a whole and/or other portions of the controller 404) are discussed below with respect to FIG. 7.

The control engine 506 may generate and process data associated with control, communication, and/or other signals sent to and obtained from the users 451 (including associated user systems 455), the sensor devices 460, the sensor devices 360, the controllers 304, the other controllers 404 of the data analysis system 450, the fluid component sources 428, the conveyance system 448, the network manager 480, and the other components of the system 400. In certain embodiments, the control engine 506 of the controller 404 may communicate with one or more components of a system external to the system 400. For example, the control engine 506 may interact with an inventory management system by ordering replacements for components or pieces of equipment (e.g., a sensor device 460, a valve 485, a motor) within the system 400 that has failed or is failing. As another example, the control engine 506 may interact with a contractor or workforce scheduling system by arranging for the labor needed to replace a component or piece of equipment in the system 400. In this way and in other ways, the controller 404 is capable of performing a number of functions beyond what could reasonably be considered a routine task.

In certain example embodiments, the control engine 506 may include an interface that enables the control engine 506 to communicate with the sensor devices 460, the sensor devices 360, the controllers 304, the other controllers 404 of the data analysis system 450, the fluid component sources 428, the conveyance system 448, the user systems 455, the network manager 480, and/or other components of the system 400. For example, if a user system 455 operates under IEC Standard 62386, then the user system 455 may have a serial communication interface that will transfer data to the controller 404. Such an interface may operate in conjunction with, or independently of, the protocols 532 used to communicate between the controller 404 and the users 451 (including corresponding user systems 455), the sensor devices 460, the sensor devices 360, the controllers 304, the other controllers 404 of the data analysis system 450, the fluid component sources 428, the conveyance system 448, the network manager 480, and the other components of the system 400.

The control engine 506 (or other components of the controller 404) may also include one or more hardware components and/or software elements to perform its functions. Such components may include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The signature generation module 545 of the controller 404 may be configured to generate one or more signatures associated with each of the samples 467 (e.g., from cuttings, from core samples, from produced fluids, from downhole oil sample) that are collected and tested. A signature may be generated by the signature generation module 545 based on the results of one or more of a number of testing methods applied to a sample 467. Examples of such testing methods may include, but are not limited to, whole oil gas chromatograph analysis, oil biomarker GC-MS analysis, stable carbon isotope analysis, stable sulfur isotope analysis, SARA, sulfur analysis, Ni/V analysis, DNA sequencing for oil samples, water analysis, alkylbenzene analysis, 2D/3D GC-MS for detailed oil component analysis, and water analysis (e.g., water wash impact, water type). In some cases, results from such testing methods may generate an oil parameter database that contains a large number (e.g., at least 20,000, at least 1 million) of measurements of parameters per sample 467.

In certain example embodiments, additionally or alternatively, the signature generation module 545 of the controller 404 may be configured to identify and filter out signatures from samples 467 that include certain compounds and/or materials that may alter results. Such signatures from certain compounds and/or materials can skew data analysis and interpretation of the overall group of signatures, and so their identification and removal when the signatures are created and organized may reduce or eliminate the risk of generating recommendations that fail to improve field operations and/or production of one or more wells 420.

For example, the signature generation module 545 may be configured to recognize signatures from samples 467 that include oil based mud (OBM), which can be found in drilling fluid that mixes with samples 467 taken from cuttings. OBM may alter the results. Specifically, the signature generation module 545 may be configured to identify and remove OBM signatures from original GC chromatograph outputs based on oil-OBM allocation results and/or to identify and remove the one or more sections that include certain compounds and/or materials that may alter results (e.g., OBM) from signatures. In certain example embodiments, the signature generation module 545 may be configured to use a customized (e.g., self-learning, user-driven) data analysis process to identify and remove signatures that include OBM and other similar compounds and/or materials.

In certain example embodiments, additionally or alternatively, the signature generation module 545 of the controller 404 may be configured to organize (e.g., combine, group, order) signatures from various samples 467 (e.g., from extracted oil, from downhole oil, from produced oil) or groups of samples from one or more wells 420. In such cases, the signature generation module 545 may be configured to implement a customized data interpretation process that organizes, using one or more protocols 532 and/or one or more algorithms 533, some or all of the signatures that are generated.

The SDHIP determination module 541 of the controller 404 may be configured to determine one or more SDHIPs for one or more of the wells 420. For example, the SDHIP determination module 541 may use one or more signatures generated by the signature generation module 545 and, in some cases, measurements of parameters taken by one or more of the sensor devices 460, where the parameters are associated with the samples 467 or portions thereof (e.g., produced water) from a well 420, to populate one or more matrices (e.g., a calibration matrix, a QA/QC matrix, a production allocation matrix) that are generated and maintained by the SDHIP determination module 541 (and in some cases also the EDHIP determination module 544). In addition, using one or more protocols 532 and/or one or more algorithms 533, the SDHIP determination module 541 may use one or more of the matrices to generate one or more SDHIPs for each well 420 (well 420-1, well 420-X).

In such cases, the SDHIP determination module 541 may be configured to implement a customized data interpretation process that locates unique oil signatures to differentiate certain samples 467 (e.g., oil samples). For example, the SDHIP determination module 541 may be configured to use a customized (e.g., self-learning, user-driven) data interpretation process to combine individual signatures and to identify unique parameters to correlate the various samples 467 (e.g., extracted oil, downhole oil, produced oil). As another example, the SDHIP determination module 541 may be configured to use a customized (e.g., self-learning, user-driven) data interpretation process to utilize production data, formation log information, well landing depth, reservoir fault analysis, and/or other surveillance data as appropriate to estimate the SDHIP.

This may be used to assist the SDHIP determination module 541 and/or the recommendation module 542 to allocate oil production from different formation layers by referencing representative oil signatures from all available samples 467. The various signatures identified and organized by the signature generation module 545 may differentiate and correlate samples 467. In some cases, the various signatures identified and organized by the signature generation module 545 may have linear correlation among calibration oil standards matrices maintained by the SDHIP determination module 541, which may allow for the eventual allocation (e.g., by the recommendation module 542) of oil production from the various formation layers.

In addition, or in the alternative, the SDHIP determination module 541 may be configured to compare one group of signatures (e.g., signatures of samples 467 obtained before production of a well 420) with another group of signatures (e.g., signatures of samples 467 obtained during production of the well 420). This comparison may be performed by the SDHIP determination module 541 before populating one or more matrices (e.g., a calibration matrix, a QA/QC matrix, a production allocation matrix).

In addition, the SDHIP determination module 541 may also be configured to modify, using one or more protocols 532, one or more algorithms 533, and/or stored data 534, an existing matrix, generate a new matrix, and/or modify a SDHIP for a well 420 using subsequent measurements of one or more parameters associated with one or more samples 467 by one or more sensor devices 460. In certain example embodiments, the SDHIP determination module 541 may be configured to use a customized (e.g., self-learning, user-driven) data interpretation process to establish and maintain standard matrices to allow for allocating oil production from the various formation layers in a subterranean formation (e.g., subterranean formation 110). In addition, or in the alternative, the SDHIP determination module 541 may be configured to use a customized (e.g., self-learning, user-driven) data interpretation process to establish and maintain uncertainty matrices for reporting data and for the QA/QC process.

Implementation of the functions of the SDHIP determination module 541 may be performed in one or more of a number of ways. For example, the SDHIP determination module 541 may determine a difference in at least one parameter between a baseline value (or range of values) and a result of testing the samples 467 or portions thereof (e.g., produced water) for at least one of the wells 420 during a field operation, where the difference exceeds a threshold value (e.g., part of the stored data 534) for the at least one parameter.

In certain example embodiments, the SDHIP determination module 541 may be configured to generate a production allocation (e.g., for oil, for water) and/or a fluid correlation of one or more wells 420 and/or among the various layers of the subterranean formation through which one or more wells 420 pass. As an example, the SDHIP determination module 541 may be configured to generate a production allocation that is applied to oil source allocation for the well 420, one or more adjacent wells 420, or any combination thereof. As another example, the SDHIP determination module 541 may be configured to generate a production allocation that is applied to water source allocation for the well 420, one or more adjacent wells 420, or any combination thereof. In such cases, the SDHIP determination module 541 may generate a production allocation and/or a fluid correlation based on comparing signatures (e.g., generated by the signature generation module 545) over time. Subsequently, the SDHIP determination module 541 may use the production allocation and a fluid correlation of a well 420, one or more protocols 532, and/or one or more algorithms 533 to generate an estimate of the SDHIP for the well 420.

The EDHIP determination module 544 of the controller 404 may be configured to determine one or more EDHIPs for one or more of the wells 420. For example, the EDHIP determination module 544 may use one or more signatures generated by the signature generation module 545 and, in some cases, measurements of parameters taken by one or more of the sensor devices 460, where the parameters are associated with the samples 467 or portions thereof (e.g., produced oil) from a well 420, to populate one or more matrices (e.g., a calibration matrix, a QA/QC matrix, a production allocation matrix) that are generated and maintained by the EDHIP determination module 544. In addition, using one or more protocols 532 and/or one or more algorithms 533, the EDHIP determination module 544 may use one or more of the matrices to generate one or more EDHIPs for each well 420 (well 420-1, well 420-X).

In such cases, the EDHIP determination module 544 may be configured to implement a customized data interpretation process that locates unique oil signatures to differentiate certain samples 467 (e.g., oil samples). For example, the EDHIP determination module 544 may be configured to use a customized (e.g., self-learning, user-driven) data interpretation process to combine individual signatures and to identify unique parameters to correlate the various samples 467 (e.g., extracted oil, downhole oil, produced oil). As another example, the EDHIP determination module 544 may be configured to use a customized (e.g., self-learning, user-driven) data interpretation process to utilize production data, formation log information, well landing depth, reservoir fault analysis, and/or other surveillance data as appropriate to estimate the EDHIP.

This may be used to assist the EDHIP determination module 544 and/or the recommendation module 542 to allocate oil production from among different formation layers by referencing representative oil signatures from all available samples 467. The various signatures identified and organized by the signature generation module 545 may differentiate and correlate samples 467. In some cases, the various signatures identified and organized by the signature generation module 545 may have linear correlation among calibration oil standards matrices managed by the EDHIP determination module 544, which may allow for the eventual allocation (e.g., by the recommendation module 542) of oil production from the various formation layers.

In addition, or in the alternative, the EDHIP determination module 544 may be configured to compare one group of signatures (e.g., signatures of samples 467 obtained before production of a well 420) with another group of signatures (e.g., signatures of samples 467 obtained during production of the well 420). This comparison may be performed by the EDHIP determination module 544 before populating one or more matrices (e.g., a calibration matrix, a QA/QC matrix, a production allocation matrix). In addition, the EDHIP determination module 544 may also be configured to modify, using one or more protocols 532, one or more algorithms 533, and/or stored data 534, an existing matrix, generate a new matrix, and/or modify an EDHIP for a well 420 using subsequent measurements of one or more parameters associated with one or more samples 467 by one or more sensor devices 460.

In some cases, one or more of the matrices of the EDHIP determination module 544 may be derived from one or more of the matrices of the SDHIP determination module 541. In addition, or in the alternative, one or more of the EDHIP values generated by the EDHIP determination module 544 may be derived from one or more of the matrices generated by the SDHIP determination module 541. Implementation of the functions of the EDHIP determination module 544 may be performed in one or more of a number of ways. For example, the EDHIP determination module 544 may determine a difference in at least one parameter between a baseline value (or range of values) and results of testing the samples 467 or portions thereof (e.g., produced oil) for at least one of the wells 420 during a field operation, where the difference exceeds a threshold parameter value (e.g., part of the stored data 534) for the at least one parameter.

In certain example embodiments, the EDHIP determination module 544 may be configured to use a customized (e.g., self-learning, user-driven) data interpretation process to establish and maintain standard matrices to allow for allocating oil production from the various formation layers in a subterranean formation (e.g., subterranean formation 110). In addition, or in the alternative, the EDHIP determination module 544 may be configured to use a customized (e.g., self-learning, user-driven) data interpretation process to establish and maintain uncertainty matrices for reporting data and for the QA/QC process. As another example, the EDHIP determination module 544 may be configured to use a customized (e.g., self-learning, user-driven) data interpretation process to utilize production data, formation log information, well landing depth, reservoir fault analysis, and/or other surveillance data as appropriate to estimate the EDHIP.

In certain example embodiments, the EDHIP determination module 544 may be configured to generate a production allocation (e.g., for oil, for water) and/or a fluid correlation of one or more wells 420 and/or among the various layers of the subterranean formation through which one or more wells 420 pass. As an example, the EDHIP determination module 544 may be configured to generate a production allocation that is applied to oil source allocation for the well 420, one or more adjacent wells 420, or any combination thereof. As another example, the EDHIP determination module 544 may be configured to generate a production allocation that is applied to water source allocation for the well 420, one or more adjacent wells 420, or any combination thereof. In such cases, the EDHIP determination module 544 may generate a production allocation and/or a fluid correlation based on comparing signatures (e.g., generated by the signature generation module 545) over time. Subsequently, the EDHIP determination module 544 may use the production allocation and a fluid correlation of a well 420, one or more protocols 532, and/or one or more algorithms 533 to generate an estimate of the EDHIP for the well 420.

The recommendation module 542 of the controller 404 may be configured to generate a recommendation regarding one or more of the wells 420. For example, the recommendation module 542 may use the output of the SDHIP determination module 541, the output of the EDHIP determination module 544, one or more protocols 532, and/or one or more algorithms 533 to generate a recommendation as to particular features (e.g., a particular fluid 437 to be used, a flow rate of the fluid 437, a pressure of the fluid 437, a particular well 420 to target, a duration, a start date and time) of a field operation, a landing depth for a well 420, spacing among multiple wells 420, a landing design (e.g., diagonal crossing, rolling cube), a production allocation (e.g., for oil, for water) of a well 420, a fluid correlation of a well 420, a way to improve oil production for a well 420, a forecast of oil related production issues for a well, and/or one or more particular wells 420 to direct a particular field operation. In some cases, the recommendation module 542 may also modify one or more of the features of a field operation that is in progress.

As another example, the recommendation module 542 may use the output of the DHIP determination module 541, the output of the EDHIP determination module 544, one or more protocols 532, and/or one or more algorithms 533 to recommend an alteration of a chemical composition of a fluid 437 (also sometimes called a field operation fluid 437) used for a field operation. As yet another example, the recommendation module 542 may use the output of the SDHIP determination module 541, the output of the EDHIP determination module 544, one or more protocols 532, and/or one or more algorithms 533 to recommend an alteration of at least one parameter (e.g., a flow rate, a pressure, a temperature) of the field operation fluid. As still another example, the recommendation module 542 may use one or more protocols 532 and/or one or more algorithms 533 to recommend a modification to an algorithm 533 to model a reservoir.

As a specific example, the recommendation module 542 may use the output of the SDHIP determination module 541, the output of the EDHIP determination module 544, one or more protocols 532, and/or one or more algorithms 533 to generate a recommended landing depth of a current or future well 420.

Implementation of the functions of the recommendation module 542 may be performed in one or more of a number of ways. For example, the recommendation module 542 may use one or more algorithms 533 and/or protocols 532 to determine that a difference between the results and the expected results exceeds a threshold forecast value. In such a case, the recommendation module 542 may use the output of the SDHIP determination module 541, the output of the EDHIP determination module 544, one or more algorithms 533, and/or protocols 532 to generate a revision to the algorithm 533 (e.g., a forecasting model) based on the difference. As another example, the recommendation module 542 may use one or more algorithms 533 and/or protocols 532 to generate a recommendation for a subsequent well 420 added to a pad of existing wells 420.

The field operation evaluation module 543 of the controller 404 may be configured to evaluate a field operation currently being performed or planned to be performed on one or more of the wells 420. For example, the operation evaluation module 543 may use the output of the SDHIP determination module 541, the output of the EDHIP determination module 544, one or more protocols 532, and/or one or more algorithms 533, as well as measurements of one or more parameters made by one or more sensor devices 460, to compare the results of testing samples 467 from a well 420 during a field operation to expected results generated by one or more algorithms 533 (e.g., a forecasting model). Any differences that exceed a threshold value may be used by the field operation evaluation module 543 as a basis of evaluating the field operation.

Implementation of the functions of the field operation evaluation module 543 may be performed in one or more of a number of ways. For example, the field operation evaluation module 543 may use the output of the SDHIP determination module 541, the output of the EDHIP determination module 544, one or more algorithms 533, and/or protocols 532 to recommend a change to a field operation based on a difference determined between a baseline (or range of baseline values) and the results of testing the samples 467 from one or more wells 420 during the field operation.

The communication module 507 of the controller 404 determines and implements the communication protocol (e.g., from the protocols 532 of the storage repository 531) that is used when the control engine 506 communicates with (e.g., sends signals to, obtains signals from) the user systems 455, the sensor devices 460, the sensor devices 360, the controllers 304, the other controllers 404 of the data analysis system 450, the fluid component sources 428, the conveyance system 448, the network manager 480, and the other components of the system 400. In some cases, the communication module 507 accesses the stored data 534 to determine which communication protocol is used to communicate with another component of the system 400. In addition, the communication module 507 may identify and/or interpret the communication protocol of a communication obtained by the controller 404 so that the control engine 506 may interpret the communication. The communication module 507 may also provide one or more of a number of other services with respect to data sent from and obtained by the controller 404. Such services may include, but are not limited to, data packet routing information and procedures to follow in the event of data interruption.

The timer 535 of the controller 404 may track clock time, intervals of time, an amount of time, and/or any other measure of time. The timer 535 may also count the number of occurrences of an event, whether with or without respect to time. Alternatively, the control engine 506 may perform a counting function. The timer 535 is able to track multiple time measurements and/or count multiple occurrences concurrently. The timer 535 may track time periods based on an Instruction obtained from the control engine 506, based on an instruction obtained from a user 451, based on an instruction programmed in the software for the controller 404, based on some other condition (e.g., the occurrence of an event) or from some other component, or from any combination thereof. In certain example embodiments, the timer 535 may provide a time stamp for each packet of data obtained from another component (e.g., a sensor device 460) of the system 400.

The power module 530 of the controller 404 obtains power from a power supply (e.g., AC mains) and manipulates (e.g., transforms, rectifies, inverts) that power to provide the manipulated power to one or more other components (e.g., the timer 535, the control engine 506) of the controller 404, where the manipulated power is of a type (e.g., alternating current, direct current) and level (e.g., 12V, 24V, 120V) that may be used by the other components of the controller 404. In some cases, the power module 530 may also provide power to one or more of the sensor devices 460.

The power module 530 may include one or more of a number of single or multiple discrete components (e.g., transistor, diode, resistor, transformer) and/or a microprocessor. The power module 530 may include a printed circuit board, upon which the microprocessor and/or one or more discrete components are positioned. In addition, or in the alternative, the power module 530 may be a source of power in itself to provide signals to the other components of the controller 404. For example, the power module 530 may be or include an energy storage device (e.g., a battery, a supercapacitor). As another example, the power module 530 may be or include a localized photovoltaic power system.

The hardware processor 521 of the controller 404 executes software, algorithms (e.g., algorithms 533), and firmware in accordance with one or more example embodiments.

Specifically, the hardware processor 521 may execute software on the control engine 506 or any other portion of the controller 404, as well as software used by the users 451 (including associated user systems 455), the network manager 480, and/or other components of the system 400. The hardware processor 521 may be an integrated circuit, a central processing unit, a multi-core processing chip, SoC, a multi-chip module including multiple multi-core processing chips, or other hardware processor in one or more example embodiments. The hardware processor 521 may be known by other names, including but not limited to a computer processor, a microprocessor, and a multi-core processor.

In one or more example embodiments, the hardware processor 521 executes software instructions stored in memory 522. The memory 522 includes one or more cache memories, main memory, and/or any other suitable type of memory. The memory 522 may include volatile and/or non-volatile memory. The memory 522 may be discretely located within the controller 404 relative to the hardware processor 521. In certain configurations, the memory 522 may be integrated with the hardware processor 521.

In certain example embodiments, the controller 404 does not include a hardware processor 521. In such a case, the controller 404 may include, as an example, one or more field programmable gate arrays (FPGA), one or more insulated-gate bipolar transistors (IGBTs), and/or one or more integrated circuits (ICs). Using FPGAs, IGBTs, ICs, and/or other similar devices known in the art allows the controller 404 (or portions thereof) to be programmable and function according to certain logic rules and thresholds without the use of a hardware processor. Alternatively, FPGAs, IGBTs, ICs, and/or similar devices may be used in conjunction with one or more hardware processors 521.

The transceiver 524 of the controller 404 may send and/or obtain control and/or communication signals. Specifically, the transceiver 524 may be used to transfer data between the controller 404 and the users 451 (including associated user systems 455), the sensor devices 460, the sensor devices 360, the controllers 304, the other controllers 404 of the data analysis system 450, the fluid component sources 428, the conveyance system 448, the network manager 480, and the other components of the system 400. The transceiver 524 may use wired and/or wireless technology. The transceiver 524 may be configured in such a way that the control and/or communication signals sent and/or obtained by the transceiver 524 may be obtained and/or sent by another transceiver that is part of a user system 455, a sensor device 460, the sensor devices 360, the controllers 304, the other controllers 404 of the data analysis system 450, the fluid component sources 428, the conveyance system 448, the network manager 480, and/or another component of the system 400. The transceiver 524 may send and/or obtain any of a number of signal types, including but not limited to radio frequency signals.

When the transceiver 524 uses wireless technology, any type of wireless technology may be used by the transceiver 524 in sending and obtaining signals. Such wireless technology may include, but is not limited to, Wi-Fi, Zigbee, VLC, cellular networking, BLE, UWB, and Bluetooth. The transceiver 524 may use one or more of any number of suitable communication protocols (e.g., ISA100, HART) when sending and/or obtaining signals.

Optionally, in one or more example embodiments, the security module 523 secures interactions between the controller 404, the users 451 (including associated user systems 455), the sensor devices 460, the sensor devices 360, the controllers 304, the other controllers 404 of the data analysis system 450, the fluid component sources 428, the conveyance system 448, the network manager 480, and the other components of the system 400. More specifically, the security module 523 authenticates communication from software based on security keys verifying the identity of the source of the communication. For example, user software may be associated with a security key enabling the software of a user system 455 to interact with the controller 404. Further, the security module 523 may restrict receipt of information, requests for information, and/or access to information.

A user 451 may be any person that interacts, directly or indirectly, with a controller 404 and/or any other component of the testing system 400. Examples of a user 451 may include, but are not limited to, a business owner, an engineer, a company representative, a geologist, a consultant, a drilling engineer, a contractor, and a manufacturer's representative. A user 451 may use one or more user systems 455, which may include a display (e.g., a GUI). A user system 455 of a user 451 may interact with (e.g., send data to, obtain data from) the controller 404 via an application interface and using the communication links 405. The user 451 may also interact directly with the controller 404 through a user interface (e.g., keyboard, mouse, touchscreen).

The network manager 480 is a device or component that controls all or a portion (e.g., a communication network, the controller 404) of the system 400. The network manager 480 may be substantially similar to some or all of the controller 404, as described above. For example, the network manager 480 may include a controller that has one or more components and/or similar functionality to some or all of the controller 404. Alternatively, the network manager 480 may include one or more of a number of features in addition to, or altered from, the features of the controller 404. As described herein, control and/or communication with the network manager 480 may include communicating with one or more other components of the same system 400 and/or another system. In such a case, the network manager 480 may facilitate such control and/or communication. The network manager 480 may be called by other names, including but not limited to a master controller, a network controller, and an enterprise manager. The network manager 480 may be considered a type of computer device, as discussed below with respect to FIG. 6.

Interaction between each controller 404, the sensor devices 460, the sensor devices 360, the controllers 304, the other controllers 404 of the data analysis system 450, the fluid component sources 428, the conveyance system 448, the users 451 (including any associated user systems 455), the network manager 480, and other components (e.g., the valves 485, the wells 420) of the system 400 may be conducted using communication links 405 and/or power transfer links 487. Each communication link 405 may include wired (e.g., Class 1 electrical cables, Class 2 electrical cables, electrical connectors, Power Line Carrier, RS485) and/or wireless (e.g., Wi-Fi, Zigbee, visible light communication, cellular networking, Bluetooth, Bluetooth Low Energy (BLE), ultrawide band (UWB), WirelessHART, ISA100) technology. A communication link 405 may transmit signals (e.g., communication signals, control signals, data) between each controller 404, the sensor devices 460, the sensor devices 360, the controllers 304, the other controllers 404 of the data analysis system 450, the fluid component sources 428, the conveyance system 448, the users 451 (including any associated user systems 455), the network manager 480, and the other components of the system 400.

Each power transfer link 487 may include one or more electrical conductors, which may be individual or part of one or more electrical cables. In some cases, as with inductive power, power may be transferred wirelessly using power transfer links 487. A power transfer link 487 may transmit power between each controller 404, the sensor devices 460, the sensor devices 360, the controllers 304, the other controllers 404 of the data analysis system 450, the fluid component sources 428, the conveyance system 448, the users 451 (including any associated user systems 455), the network manager 480, and the other components of the system 400. Each power transfer link 487 may be sized (e.g., 12 gauge, 18 gauge, 4 gauge) in a manner suitable for the amount (e.g., 480V, 24V, 120V) and type (e.g., alternating current, direct current) of power transferred therethrough.

Each of the controllers 304 is a device or component that controls a portion (e.g., a communication network, some of the field equipment 109) of the system 400. A controller 304 may be substantially similar to some or all of the controller 404, as described above. For example, a controller 304 may include a controller that has one or more components and/or similar functionality to some or all of the controller 404. Alternatively, a controller 304 may include one or more of a number of features in addition to, or altered from, the features of the controller 404.

As described herein, control and/or communication with a controller 304 may include communicating with one or more other components of the same system 400 and/or another system. In such a case, a controller 304 may facilitate such control and/or communication. Each controller 304 may be considered a type of computer device, as discussed below with respect to FIG. 6.

Each sensor device 360 of the system 400 may be substantially the same as a sensor device 460 of the data analysis system 450. For example, each sensor device 360 includes one or more sensors that measure one or more parameters (e.g., pressure, flow rate, temperature, humidity, fluid content, voltage, current, permeability, porosity, rock characteristics, chemical elements in a fluid, chemical elements in a solid, concentrations, etc.). Examples of a sensor of a sensor device 360 may include, but are not limited to, a temperature sensor, a flow sensor, a pressure sensor, a gas spectrometer, a voltmeter, an ammeter, a permeability meter, a spectrograph, a gas chromatograph a porosimeter, and a camera. A sensor device 360 may be a stand-alone device or integrated with another component of the system 400. When a sensor device 360 includes a controller, the sensor device 360 may correspond to a computer system as described below with regard to FIG. 6.

A user 451 (which may include an associated user system 455), the sensor devices 460, the sensor devices 360, the controllers 304, the other controllers 404 of the data analysis system 450, the fluid component sources 428, the conveyance system 448, the network manager 480, and the other components of the system 400 may interact with a controller 404 using the application interface 526. Specifically, the application interface 526 of a controller 404 obtains data (e.g., information, communications, instructions, updates to firmware) from and sends data (e.g., information, communications, instructions) to the user systems 455 of the users 451, the sensor devices 460, the sensor devices 360, the controllers 304, the other controllers 404 of the data analysis system 450, the fluid component sources 428, the conveyance system 448, the network manager 480, and/or the other components of the system 400. Examples of an application interface 526 may be or include, but are not limited to, an application programming interface, a web service, a data protocol adapter, some other hardware and/or software, or any suitable combination thereof. Similarly, the user systems 455 of the users 451, the sensor devices 460, the sensor devices 360, the controllers 304, the other controllers 404 of the data analysis system 450, the fluid component sources 428, the conveyance system 448, the network manager 480, and/or the other components of the system 400 may include an interface (similar to the application interface 526 of the controller 404) to obtain data from and send data to a controller 404 in certain example embodiments.

In addition, as discussed above with respect to a user system 455 of a user 451, one or more of the sensor devices 460, one or more of the sensor devices 360, one or more of the controllers 304, one or more of the other controllers 404 of the data analysis system 450, one or more of the fluid component sources 428, some or all of the conveyance system 448, the network manager 480, and/or one or more of the other components (or portions thereof) of the system 400 may include a user interface. Examples of such a user interface may include, but are not limited to, a graphical user interface, a touchscreen, a keyboard, a monitor, a mouse, some other hardware, or any suitable combination thereof.

The controller 404, the users 451 (including associated user systems 455), the sensor devices 460, the sensor devices 360, the controllers 304, the other controllers 404 of the data analysis system 450, the fluid component sources 428, the conveyance system 448, the network manager 480, and the other components of the system 400 may use their own system or share a system in certain example embodiments. Such a system may be, or contain a form of, an Internet-based or an intranet-based computer system that is capable of communicating with various software. A computer system includes any type of computing device and/or communication device, including but not limited to a controller 404. Examples of such a system may include, but are not limited to, a desktop computer with a Local Area Network (LAN), a Wide Area Network (WAN), Internet or intranet access, a laptop computer with LAN, WAN, Internet or intranet access, a smart phone, a server, a server farm, an android device (or equivalent), a tablet, smartphones, and a personal digital assistant (PDA). Such a system may correspond to a computer system as described below with regard to FIG. 6.

Further, as discussed above, such a system may have corresponding software (e.g., user system software, sensor device software, controller software). The software may execute on the same or a separate device (e.g., a server, mainframe, desktop personal computer (PC), laptop, PDA, television, cable box, satellite box, kiosk, telephone, mobile phone, or other computing devices) and may be coupled by the communication network (e.g., Internet, Intranet, Extranet, LAN, WAN, or other network communication methods) and/or communication channels, with wire and/or wireless segments according to some example embodiments. The software of one system may be a part of, or operate separately but in conjunction with, the software of another system within the system 400.

FIG. 6 illustrates one embodiment of a computing device 618 that implements one or more of the various techniques described herein, and which is representative, in whole or in part, of the elements described herein pursuant to certain example embodiments. For example, a controller 404 (including components thereof, such as a control engine 506, a hardware processor 521, a storage repository 531, a power module 530, and a transceiver 524) may be considered a computing device 618 (also called a computer system 618 herein). Computing device 618 is one example of a computing device and is not intended to suggest any limitation as to scope of use or functionality of the computing device and/or its possible architectures. Neither should the computing device 618 be interpreted as having any dependency or requirement relating to any one or combination of components illustrated in the example computing device 618.

The computing device 618 includes one or more processors or processing units 614, one or more memory/storage components 615, one or more input/output (I/O) devices 616, and a bus 617 that allows the various components and devices to communicate with one another. The bus 617 represents one or more of any of several types of bus structures, including a memory bus or memory controller, a peripheral bus, an accelerated graphics port, and a processor or local bus using any of a variety of bus architectures. The bus 617 includes wired and/or wireless buses.

The memory/storage component 615 represents one or more computer storage media. The memory/storage component 615 includes volatile media (such as random access memory (RAM)) and/or nonvolatile media (such as read only memory (ROM), flash memory, optical disks, magnetic disks, and so forth). The memory/storage component 615 includes fixed media (e.g., RAM, ROM, a fixed hard drive, etc.) as well as removable media (e.g., a Flash memory drive, a removable hard drive, an optical disk, and so forth).

One or more I/O devices 616 allow a user 451 to enter commands and information to the computing device 618, and also allow information to be presented to the user 451 and/or other components or devices. Examples of input devices 616 include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques are stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 618 is connected to a network (not shown) (e.g., a LAN, a WAN such as the Internet, cloud, or any other similar type of network) via a network interface connection (not shown) according to some example embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other example embodiments. Generally speaking, the computer system 618 includes at least the minimal processing, input, and/or output means necessary to practice one or more embodiments.

Further, those skilled in the art will appreciate that one or more elements of the aforementioned computer device 618 is located at a remote location and connected to the other elements over a network in certain example embodiments. Further, one or more embodiments is implemented on a distributed system having one or more nodes, where each portion of the implementation (e.g., a fluid component source 428, a testing apparatus 470, the sample processing system 495) is located on a different node within the distributed system. In one or more embodiments, the node corresponds to a computer system. Alternatively, the node corresponds to a processor with associated physical memory in some example embodiments. The node alternatively corresponds to a processor with shared memory and/or resources in some example embodiments.

FIG. 7 shows a flowchart 758 of a method for] according to certain example embodiments. While the various steps in this flowchart 758 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order. Some or all of the steps of the method of FIG. 7 may be performed off site (e.g., in a laboratory remote from a field operation). In addition, or in the alternative, some or all of the steps of the method of FIG. 7 may be performed on site (e.g., in the field, adjacent to a wellbore 120) where a field operation is being performed or planned.

In addition, a person of ordinary skill in the art will appreciate that additional steps not shown in FIG. 7 may be included in performing this method. Accordingly, the specific arrangement of steps should not be construed as limiting the scope. Further, a particular computing device, such as the computing device 618 discussed above with respect to FIG. 6, may be used to facilitate (e.g., direct, control, provide instructions, provide recommendations, perform, execute) the performance of one or more of the steps for the methods shown in FIG. 7 in certain example embodiments. Any of the functions performed below by a controller 404 (an example of which is shown in FIG. 5) may involve the use of one or more protocols 532, one or more algorithms 533, and/or stored data 534 stored in a storage repository 531. In addition, or in the alternative, any of the functions in the method may be performed by a user (e.g., user 451).

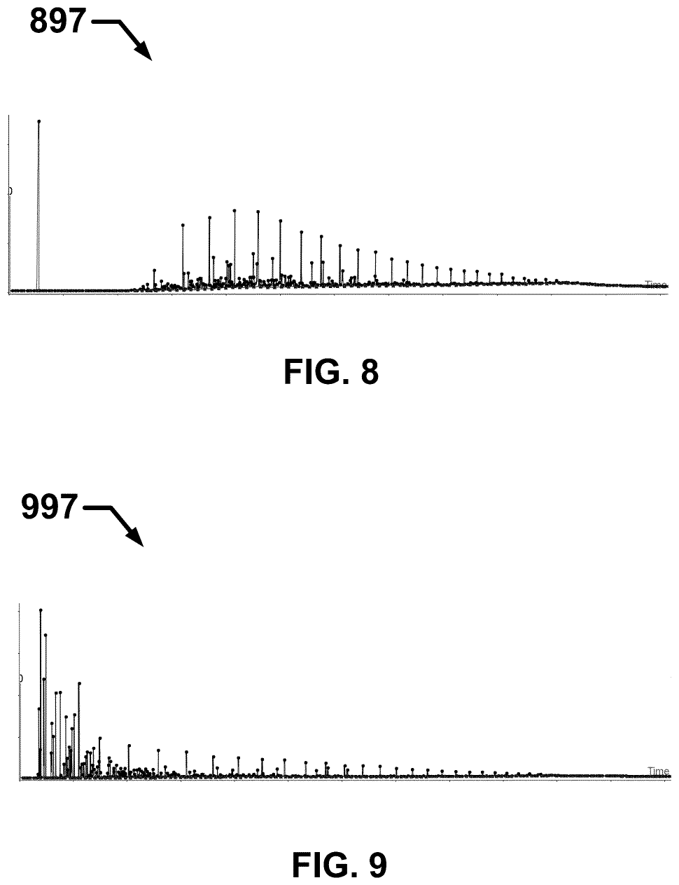

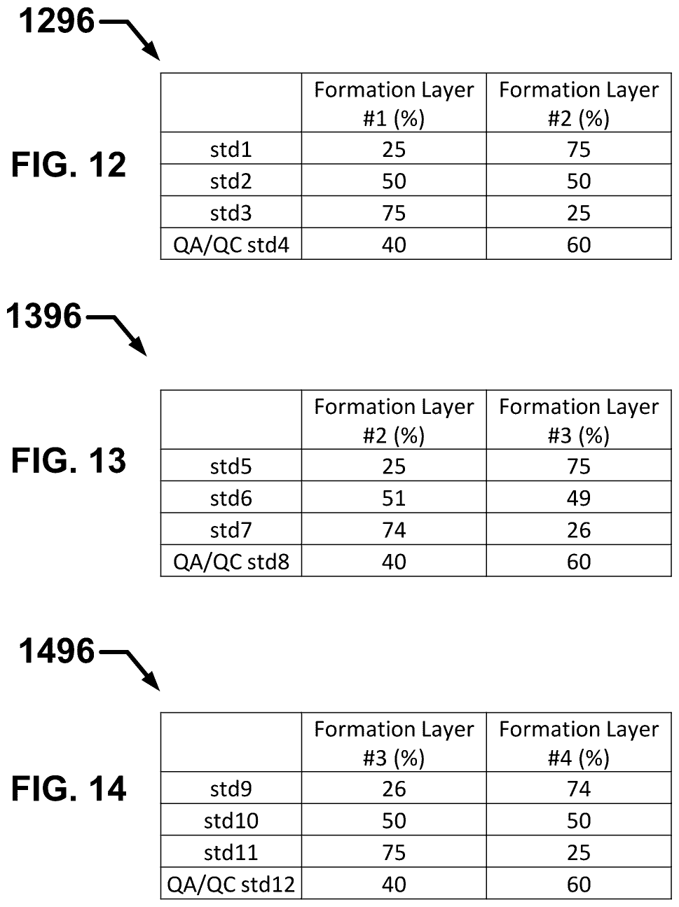

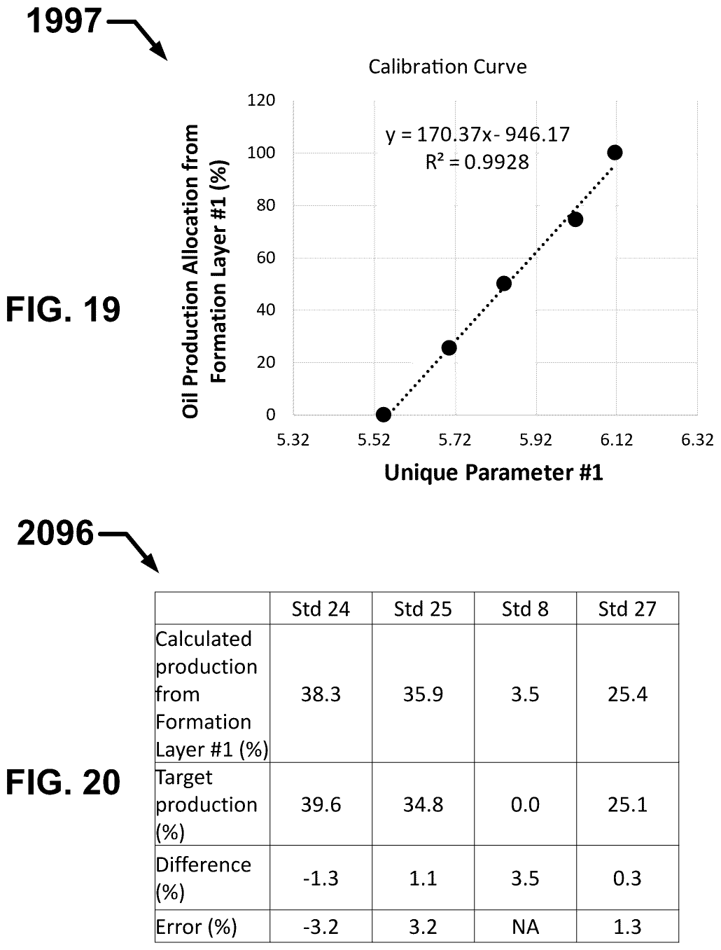

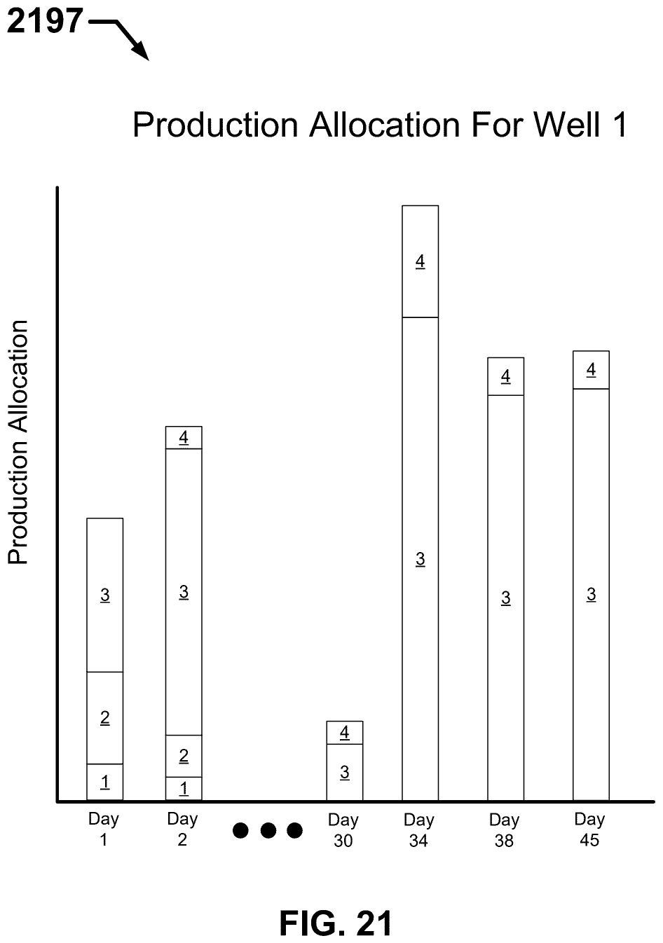

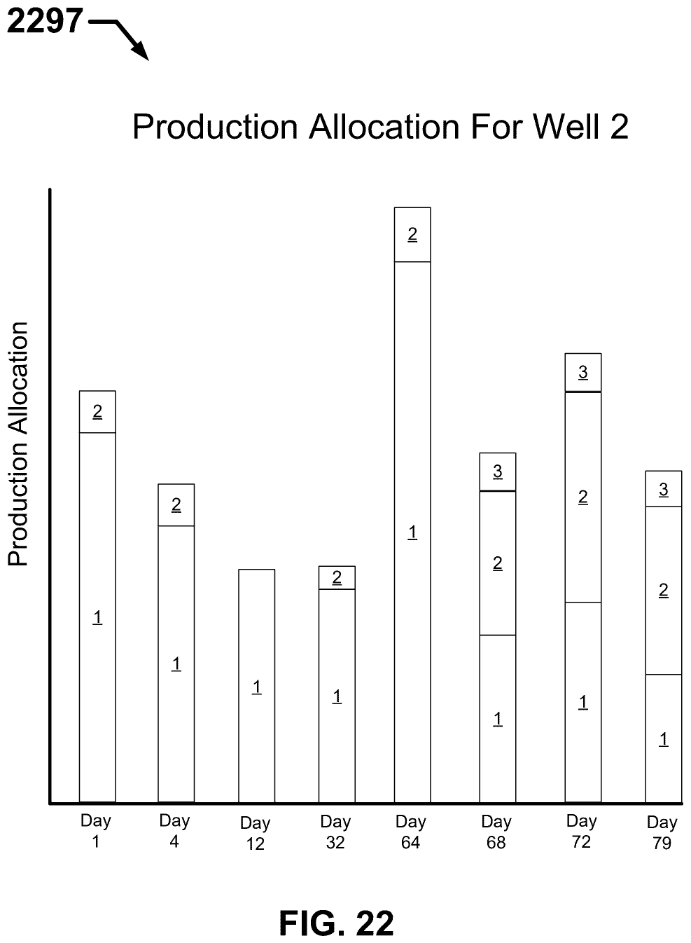

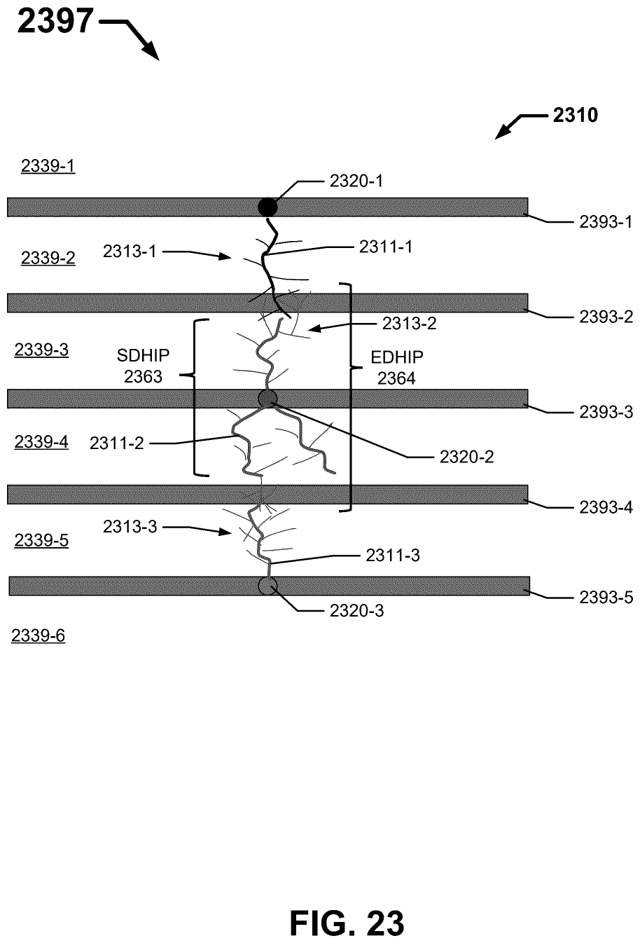

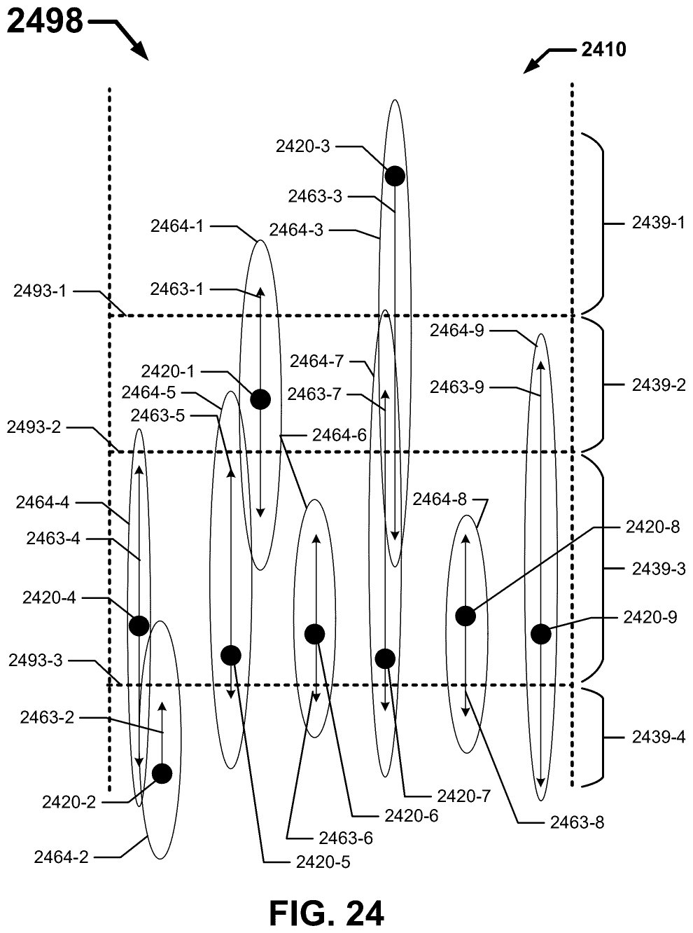

The method shown in FIG. 7 is merely an example that may be performed by using an example system described herein. In other words, systems for estimating a SDHIP for a well drilled into multiple subterranean formations may perform other functions using other methods in addition to and/or aside from those shown in FIG. 7. FIGS. 8 through 11 show graphs that illustrate measurements of samples 467 that are used to generate signatures for the samples 467 according to certain example embodiments. FIGS. 12 through 18 show examples of tables used to generate matrices of signatures according to certain example embodiments. FIG. 19 shows a graph illustrating production allocation for a formation according to certain example embodiments. FIG. 20 shows an example of a table used to determine QA/QC according to certain example embodiments. FIGS. 21 and 22 show examples of graphs illustrating production allocation for wells 420 according to certain example embodiments. FIG. 23 shows a graph of SDHIP and EDHIP ranges of a well 420 through several formations of a subterranean formation according to certain example embodiments. FIG. 24 shows a gun barrel diagram of multiple wells in a subterranean formation for use with certain example embodiments.

Referring to the description above with respect to FIGS. 1A through 6, the method shown in the flowchart 758 of FIG. 7 begins at the START step and proceeds to step 781, where samples 467 for one or more wells 420 are obtained. As used herein, the term “obtaining” may include collecting, receiving, retrieving, accessing, generating, etc. or any other manner of obtaining samples 467 of the water from a well 420. The samples 467 may be obtained from some or all wells 420 of a pad. The samples 467 are obtained during a particular field operation. A sample 467 may include some amount of oil (e.g., produced oil, downhole oil, extracted oil). Each sample 467 may originate from a known range of depths or locations in the well 420.

Each sample 467 may be broadly categorized as being obtained at a point in time relative to a field operation (e.g., during drilling, during fracturing, during shut-in, during production) performed on the well 420 and/or on one or more adjacent wells 420. For example, during one period of time, samples 467 may be obtained before production of a well 420. During a subsequent period of time, additional samples 467 may be obtained during production of the well 420. In such cases, the samples 467 may include produced fluid.

Each sample 467 may be extracted from a wellbore 420 during a part (e.g., exploration, production, shut-in period) of a field operation. The samples 467 may be obtained at the surface (e.g., surface 108, surface 208) outside the well 420 during a known point in time or period of time using field equipment 109, part of the conveyance system 448, and/or other equipment (e.g., pumps, compressors). Samples 467 may be or include core samples, cuttings, downhole fluids, produced fluids, some other type of sample, or any combinations of these. Some or all of the process of obtaining the samples 467 from a well 420 may be controlled by a controller 404 (or a collecting component thereof) of the data analysis system 450 using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining the samples 467 from a well 420 may be controlled by a user 451.

The samples 467 may be obtained from a well 420 continuously over an extended period of time or on an iterative basis. The rate (e.g., daily, weekly, randomly) of collecting and testing samples 467 may vary (e.g., based on field operations, based on field conditions, based on business need, based on whether there is no significant change in the analyzed oil signature) over time. Samples 467 from the same well 420 and/or different wells 420 may be obtained from different formation depths (e.g., as end members). For some well networks, there may be existing (parent) wells 420 that are on production and other (child) wells 420 that have not yet been placed on production (e.g., planned wells 420, wells 420 undergoing field operations related to exploration, wells 420 awaiting a fracturing operation). In such cases, this step 781, as well as one or more other steps in this process, may apply to the some or all of the parent wells 420 and/or some or all of the child wells 420 that exist.

In step 782, one or more measurements of one or more parameters associated with the samples 467 are obtained. Measuring the one or more parameters associated with the samples 467 may be conducted using one or more sensor devices 460 to measure the one or more parameters that are directly or indirectly associated with the samples 467. The parameters associated with the samples 467 may be measured at the surface (e.g., surface 108, surface 208). The parameters associated with the samples 467 that are measured may include, but are not limited to, the composition of the samples 467, the amount (concentration) of each part of the composition, the state (e.g., liquid, solid) of each part of the composition, the temperature of the samples 467, the intensity of certain elements and/or compounds found in the samples 467, and the viscosity of the samples 467.

Measurements of one or more parameters of a sample 467 may be based on the results of one or more of a number of testing methods applied to a sample 467. Examples of such testing methods may include, but are not limited to, whole oil gas chromatograph analysis, oil biomarker GC-MS analysis, stable carbon isotope analysis, stable sulfur isotope analysis, SARA, sulfur analysis, Ni/V analysis, DNA sequencing for oil samples, water analysis, alkylbenzene analysis, 2D/3D GC-MS for detailed oil component analysis, and water analysis. In some cases, results from such testing methods may generate an oil parameter database that contains a large number (e.g., at least 20,000, at least one million) of measurements of parameters per sample 467.

Some or all of the process of measuring the parameters associated with the samples 467 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of measuring the parameters associated with the samples 467 may be controlled by a user 451. The parameters associated with the samples 467 may be measured continuously over an extended period of time or on a discrete basis.

In some cases, the samples 467 may be processed by the sample processing system 495 before being tested and/or after being tested. In the latter case, the parameters associated with the samples 467 may be retested after the samples 467 has been processed. The samples 467 may be processed multiple times and/or tested multiple times. The samples 467 may be processed for any of a number of purposes, including but not limited to removing cuttings and other unwanted solids, removing OBM, locating more representative samples 467). The samples 467 may be processed using any of a number of appropriate equipment of the sample processing system 495, including but not limited to heaters, chillers, mixers, filters, agitators, pumps, and centrifuges.

Some or all of the processing of the samples 467 using the sample processing system 495 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the processing of the samples 467 using the sample processing system 495 may be controlled by a user 451. The samples 467 may be processed using the sample processing system 495 continuously over an extended period of time or on a discrete basis.

By way of example, FIGS. 8 through 11 show graphs that illustrate measurements of samples 467 that are used to generate signatures for the samples 467 according to certain example embodiments. The graph 897 of FIG. 8 shows the results of a chemical extraction test on a sample 467 in the form of a core sample. The horizontal axis of the graph 897 is for intensity of peak, and the horizontal axis of the graph 897 is for time. The graph 997 of FIG. 9 shows the results of a test on a sample 467 in the form of produced oil for the same well as the core sample of FIG. 8 was extracted. The horizontal axis of the graph 997 is for intensity of peak, and the horizontal axis of the graph 997 is for time.

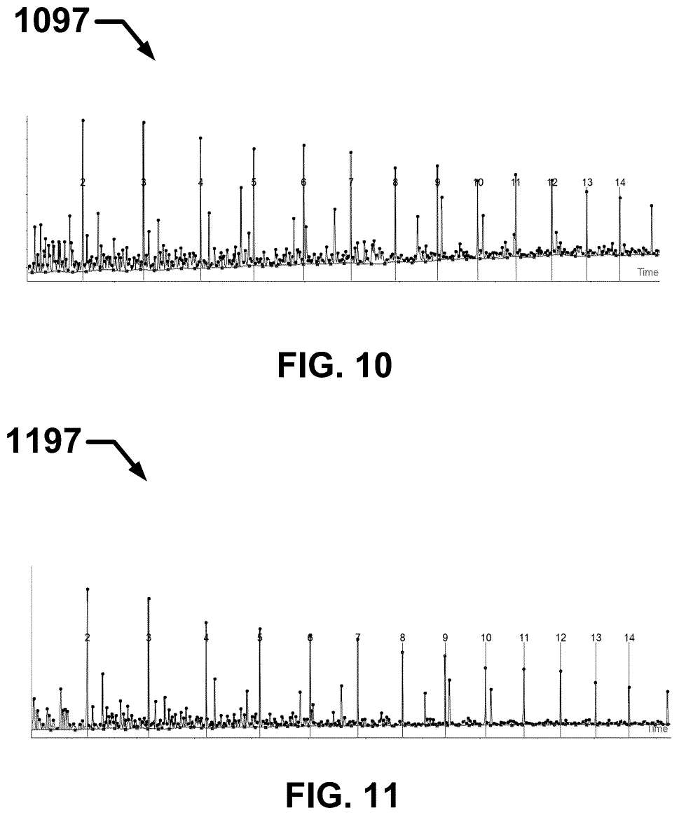

The graph 1097 of FIG. 10 shows the results of a thermal extraction test on a sample 467 in the form of a core sample. The horizontal axis of the graph 1097 is for intensity of peak, and the horizontal axis of the graph 1097 is for time. The graph 1197 of FIG. 11 shows the results of a test on a sample 467 in the form of produced oil for the same well as the core sample of FIG. 10 was extracted. The horizontal axis of the graph 1197 is for intensity of peak, and the horizontal axis of the graph 1197 is for time.

In step 783, one or more signatures of each sample 467 of the one or more wells 420 is generated. Some or all of the signatures may be generated by a controller 404 (or the signature generation module 545 thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of generating a signature for samples 467 from a well 420 may be controlled by a user 451.

The signatures for the samples 467 from a well 420 may be generated based on raw measurements made by one or more of the sensor devices 460, adjusted measurements made by one or more of the sensor devices 460, outputs of algorithms 533 (e.g., models) using measurements made by one or more of the sensor devices 460 as inputs, some other information associated with measurements made by one or more of the sensor devices 460, or any suitable combination thereof. Each signature may be specifically and/or broadly categorized (e.g., by period of time that the associated sample 467 originated from a well 420, by well 420 or origin for the associated sample 467, by subterranean formation of origin for the well 420 for the associated sample 467, by the field data and operation/events (e.g., production profile, formation log information, well landing depth, reservoir fault analysis, lithology, geology, stress gradience, pressure gradience, and other surveillance data; during drilling, during fracturing, during shut-in, before production, during production, hydrocarbon gas injection, sour gas injection, water flooding, steam flooding, polymer flooding, chemical flooding, installation of new artificial lift pumps, etc.) performed on the well 420 and/or on one or more adjacent wells 420 when the associated sample 467 originates from the well 420).

For example, during one period of time (e.g., before production of a current well 420, during production of a current well 420, before production of an adjacent well 420, during production of an adjacent well 420), samples 467 may be obtained before production of a well 420. During a subsequent period of time (e.g., during production of a current well 420, before production of one or more adjacent wells 420, during production of one or more adjacent wells 420), additional samples 467 may be obtained during production of the well 420. In such cases, the samples 467 may include produced fluid.

In certain example embodiments, the signature generation module 545 of the controller 404 may be configured to combine signatures from various samples 467 (e.g., extracted oil, downhole oil, produced oil). For example, use of a customized data interpretation process may locate unique signatures to differentiate among multiple samples 467 and eventually allocate (e.g., using the DHIP determination module 541 and/or the EDHIP determination module 544) oil production from different formations by referencing representative oil signatures from all available samples 467. Unique oil signatures may differentiate and correlate oil samples 467 and produced oil samples 467. In addition, or in the alternative, unique oil signatures may have a substantially linear correlation (e.g., as shown in FIG. 19 below) among a calibration oil standards matrix that allows for allocating oil production from a number (e.g., 1, 3, 5, 8, 15, 25, 50, 4000, 80000) of formation layers within a subterranean formation.

In certain example embodiments, as discussed above, the signature generation module 545 of the controller 404 may be configured to identify and filter out signatures from samples 467 that include OBM and other similar compounds or materials that may alter results as part of this step 783. The signature generation module 545 may be configured to use a customized (e.g., self-learning, user-driven) data analysis process to identify and remove signatures that include OBM and the like that may alter results. Also, as part of this step 783, the signature generation module 545 of the controller 404 may be configured to organize (e.g., combine, group, order) signatures from various samples 467 (e.g., from extracted oil, from downhole oil, from produced oil).

For example, the signature generation module 545 of the controller 404 may be configured to combine representative oil signatures from extracted oil, downhole oil, and produced oil to 1) identify unique oil signatures to differentiate oil samples, 2) prepare a standard matrix for production allocation that allows for allocating production (e.g., for oil, for water) from a large number of formation layers; and 3) correlate production (e.g., for oil, for water) from different formation layers and/or adjacent wells 420. Using example embodiments, combining representative oil signatures from samples 467 of different sources (e.g., cuttings, core samples, extracted oil, downhole oil, and produced oil), a customized data interpretation process may locate unique oil signatures to differentiate oil samples and allocate oil production from different formation layers and/or wells 420 by referencing representative oil signatures from all available samples 467. Unique oil signatures generated herein may differentiate and correlate oil samples and produced oil samples. In addition, or in the alternative, oil signatures generated herein may have a linear correlation among a calibration oil standards matrix, which allows for allocating oil production from a number (e.g., 1, 5, 10, 23, 55, 129, 2000, 10000) of formation layers within a subterranean formation (e.g., subterranean formation 108, subterranean formation 208).

In such cases, the signature generation module 545 may be configured to implement a customized data interpretation process that locates unique oil signatures to differentiate certain samples 467 (e.g., oil samples). For example, the signature generation module 545 may be configured to use a customized (e.g., self-learning, user-driven) data interpretation process to combine and/or group individual signatures and to identify unique parameters to correlate the signatures of various samples 467 (e.g., extracted oil, downhole oil, produced oil). Generating a signature for one or more of the samples 467 from a well 420 may be performed at the surface (e.g., surface 108, surface 208). The signatures for one or more of the samples 467 of a well 420 may be generated continuously over an extended period of time or on a discrete basis.

In step 784, a determination is made as to whether there is another group of signatures. Another group of signatures may be for the same well 420 but for a different period of time corresponding to a different stage of the well 420. For example, one group of signatures may be for a period of time before production of a well 420, and another group of signatures may be for a subsequent period of time during production of the same well 420. In addition, or in the alternative, another group of signatures may be for a different well 420 (e.g., an adjacent well 420) that may have some amount of fluidic communication with the well 420 through one or more layers of the subterranean formation (e.g., subterranean formation 110, subterranean formation 210).

The determination may be made by a controller 404 (or the SDHIP determination module 541 and/or the EDHIP determination module 544 thereof) using one or more protocols 532, one or more algorithms 533 (e.g., models), input from a user 451 (which may include an associated user system 455), and/or any other source of information within the system 400. In addition, or in the alternative, the determination may be made by a user 451. The determination may be made at the surface (e.g., surface 108, surface 208). If there is another group of signatures, then the process proceeds to step 786. If there is not another group of signatures, then the process reverts to step 781.

In step 786, different groups of signatures for one or more of the wells 420 are compared. The groups of signatures may be obtained from the signature generation module 545 and/or the storage repository 531. The groups of signatures may be obtained at the surface (e.g., surface 108, surface 208). Some or all of the process of obtaining and comparing the groups of signatures from one or more wells 420 may be performed by a controller 404 (or the DHIP determination module 541 and/or the EDHIP determination module 544) of the data analysis system 450 using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining and comparing the groups of signatures from one or more wells 420 may be performed by a user 451.

This step 786 may include considering data outside of the signatures. For example, the SDHIP determination module 541 and/or the EDHIP determination module 544 of the controller 404 may combine the field data and operation/events (e.g., production profile, formation log information, well landing depth, reservoir fault analysis, lithology, geology, stress gradience, pressure gradience, and other surveillance data; during drilling, during fracturing, during shut-in, before production, during production, hydrocarbon gas injection, sour gas injection, water flooding, steam flooding, polymer flooding, chemical flooding, installation of new artificial lift pumps, etc.) In some cases, the specific circumstances of a well 420 or grouping of wells 420 may help determine the other data that is to be used in certain example embodiments.

The groups of signatures may be obtained and/or compared continuously over an extended period of time or on an iterative basis. The rate (e.g., daily, weekly, randomly) of obtaining and/or comparing groups of signatures may vary (e.g., based on field operations, based on field conditions, based on business need, based on the existence and/or status of adjacent wells 420) over time. Groups of signatures from the same well 420 and/or different wells 420 may be obtained from different formation depths (e.g., as end members) and/or during different phases of a field operation for one or more of the wells 420. In some cases, comparing groups of signatures may result in updating one or more algorithms 533 (e.g., a model) that are used to generate one or more of the signatures.

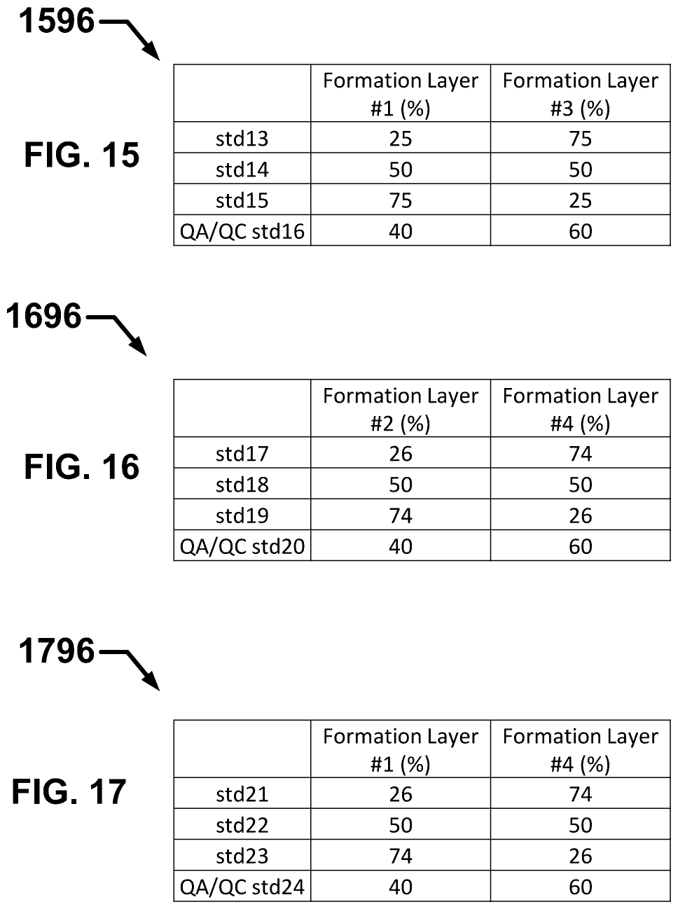

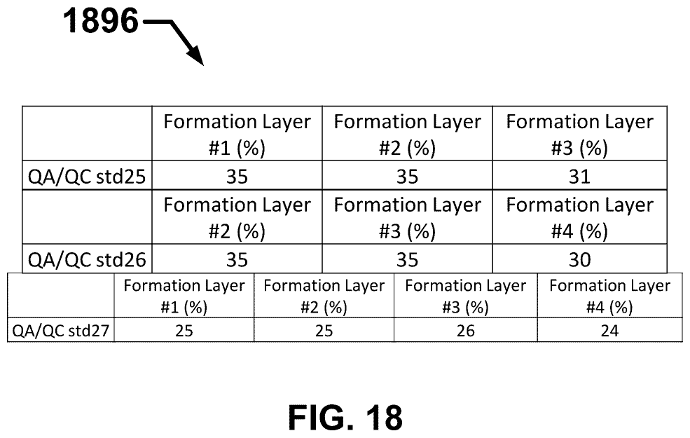

In some cases, each group of signatures may be used to populate one or more matrices. In such cases, each matrix may be generated by the SDHIP determination module 541 and/or the EDHIP determination module 544 of the controller 404 of the data analysis system 450. The matrices may be used in comparing two or more groups of signatures. FIGS. 12 through 18 show a number of example tables (table 1296 of FIG. 12, table 1396 of FIG. 13, table 1496 of FIG. 14, table 1596 of FIG. 15, table 1696 of FIG. 16, table 1796 of FIG. 17, and table 1896 of FIG. 18) that represent how some matrices may be generated. While the examples in these figures are based only on oil, in alternative embodiments one or more of the matrices (e.g., made for calibration, made for QA/QC) may additionally or alternatively be derived from other types of samples 467 (e.g., cuttings, core samples).

For example, samples 467 in the form of core samples, cuttings, and produced fluids are analyzed to generate signatures based on measurements of parameters of the samples 467 by one or more sensor devices 460 of the data analysis system 450. The tables of FIGS. 12 through 18 have rows for standards (averages), including standards for QA/QC values, and the columns are for production allocation (as a percentage) by formation layer (a total of four in this example) within a subterranean formation. Using these tables, the SDHIP determination module 541 and/or the EDHIP determination module 544 of the controller 404 may generate calibration and QA/QC standard matrices by manually mixing the signatures of samples 467 from various formation layers with different combination and ratios. For example, referring to the graph 1296 of FIG. 12, for creating std1 (an average), an amount of formation layer #1 oil/solids (i.e., oil in a sample 467 from formation layer #1) is mixed with an amount of formation layer #2 oil/solids to result in a 25.0 Wt. % of formation layer #1/75.0 Wt. % of formation layer #2 mixture.

In step 787, a production allocation and/or a fluid correlation of one or more wells 420 is generated. The production allocation (e.g., for oil, for water) and/or the fluid correlation may be generated by the SDHIP determination module 541 and/or the EDHIP determination module 544 of the controller 404. The SDHIP determination module 541 and/or the EDHIP determination module 544 may use some or all of the matrices discussed above to generate the production allocation and/or the fluid correlation. Some or all of the process of generating the production allocation and/or the fluid correlation for one or more wells 420 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of generating the production allocation and/or the fluid correlation for one or more wells 420 may be controlled by a user 451. The production allocation and/or the fluid correlation for one or more wells 420 may be generated continuously over an extended period of time or on a discrete basis.

As an example, FIG. 19 shows a graph 1997 of in which a calibration curve is established using calibration standards (averages) from one or more matrices developed using signatures. In this case, the calibration curve is a line defined by the formula shown in the graph 1997. The data points in the graph 1997 from which the calibration curve is derived are individual plots of oil production allocation from a formation layer (formation layer #1) as a percentage along the vertical axis versus one of the parameters associated with the samples 467 and measured by the sensor devices 460 along the horizontal axis. The calibration curve can therefore yield a production allocation amount when a value of the parameter is known. Further, the table 2096 of FIG. 20 provides the QA/QC validation results by showing the accuracy of the calibration curve derived from the graph 1997 of FIG. 19 for four calibration standards. As discussed above, example embodiments can be used to generate production allocation and/or fluid correlation across a network of wells 420. As an example, FIG. 21 shows a graph 2197 that conveys the overall oil production (e.g., actual, forecast) from a particular well 420 over time. Further, for each day the graph 2197 shows the amount (e.g., actual, forecast) of oil that originates from each of 4 formation layers of the subterranean formation (e.g., subterranean formation 110, subterranean formation 210) through which the well 420 passes. Similarly, FIG. 22 shows a graph 2297 that conveys the overall oil production (e.g., actual, forecast) from another well 420 (e.g., adjacent to the well 420 of FIG. 21) over time. Further, for each day the graph 2297 shows the amount (e.g., actual, forecast) of oil that originates from each of 4 formation layers of the subterranean formation (e.g., subterranean formation 110, subterranean formation 210) through which the well 420 passes. In this case, another adjacent well 420 is put on production between day 32 and day 64, which results in a change in the overall production volume and the production allocation for the well 420.

In step 788, an estimate of SDHIP and/or EDHIP for one or more wells 420 is generated. The SDHIP and/or EDHIP may be generated by the SDHIP determination module 541 and/or the EDHIP determination module 544, respectively, of the controller 404. The SDHIP determination module 541 and/or the EDHIP determination module 544 may be based on the production allocation and the fluid correlation of one or more of the wells 420. Some or all of the process of generating the estimate of the SDHIP and/or EDHIP for one or more wells 420 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of generating the estimate of the SDHIP and/or EDHIP for one or more wells 420 may be controlled by a user 451. The SDHIP and/or EDHIP for one or more wells 420 may be generated continuously over an extended period of time or on a discrete basis. In some cases, the EDHIP for a well 420 is at least as large as the SDHIP for that well 420. In some cases, the SDHIP for a well 420 may be used to estimate an original fracture in place and a generated fracture in place for the well 420.

As an example, the graph 2397 of FIG. 23 shows a subterranean formation 2310 having six formation layers 2339 (formation layer 2339-1, formation layer 2339-2, formation layer 2339-3, formation layer 2339-4, formation layer 2339-5, and formation layer 2339-6) and three wellbores 2320 (wellbore 2320-1, wellbore 2320-2, and wellbore 2320-3). There is a boundary 2393 between each pair of adjacent formation layers 2339. Specifically, boundary 2393-1 is between formation layer 2339-1 and formation layer 2339-2. Boundary 2393-2 is between formation layer 2339-2 and formation layer 2339-3. Boundary 2393-3 is between formation layer 2339-3 and formation layer 2339-4. Boundary 2393-4 is between formation layer 2339-4 and formation layer 2339-5. Boundary 2393-5 is between formation layer 2339-5 and formation layer 2339-6.

Wellbore 2320-1 in the graph 2397 is disposed largely in boundary 2393-1. Emanating from the wellbore 2320-1 into formation layer 2339-2 and the top part of formation layer 2339-3 are primary fractures 2311-1, and emanating from the primary fractures 2311-1 are secondary fractures 2313-1. Wellbore 2320-2 in the graph 2397 is disposed largely in boundary 2393-3. Emanating from the wellbore 2320-2 into formation layer 2339-3 and formation layer 2339-4 are primary fractures 2311-2, and emanating from the primary fractures 2311-2 are secondary fractures 2313-2. Wellbore 2320-3 in the graph 2397 is disposed largely in boundary 2393-5. Emanating from the wellbore 2320-3 into formation layer 2339-5 are primary fractures 2311-3, and emanating from the primary fractures 2311-3 are secondary fractures 2313-3.

Based on determining the production allocation (e.g., for oil, for water) and/or the fluid correlation of the various wells 2320, as discussed above in this method, an estimate of the SDHIP and/or the EDHIP of each well 2320 may be generated. For example, as shown in the graph 2397 in FIG. 23, well 2320-2 has a SDHIP 2363 that is substantially equal to the vertical depth of the primary fractures 2311-2 that emanate from well 2320-2. Also, well 2320-2 has an EDHIP 2364 that is substantially equal to the vertical depth of the secondary fractures 2313-2 that emanate from the primary fractures 2311-2. In some cases, a large SDHIP 2363 is desired when hydraulic fractures are connected with faults/lineaments/natural fractures, and the well can potentially drain oil from long distances. Such long-distance draining can potentially range from short term (e.g., less than 1 week) to longer term (e.g., 3 months, 1 year, more than one year).

In step 789, a recommendation for one or more of the wells 420 is generated. A recommendation may be generated by the recommendation module 542 of the controller 404. The recommendation module 542 may generate a recommendation based on the production allocation, the fluid correlation, the SDHIP, and/or the ISHIP of one or more of the wells 420. Some or all of the process of generating a recommendation for one or more wells 420 may be controlled by a controller 404 (or a collecting component thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of generating a recommendation for one or more wells 420 may be controlled by a user 451. A recommendation for one or more wells 420 may be generated continuously over an extended period of time or on a discrete basis.

A recommendation generated by the recommendation module 542 may be directed to any aspect of one or more of the wells 420. For example, a recommendation may be directed to a field operation (e.g., how one or more of the wells 420 is produced, how one or more of the wells 420 is fractured, where to land a well 420) of a well 420. As another example, a recommendation may be directed to the timing (e.g., a start time, a duration of time, and end time) of a field operation or portion thereof. As yet another example, a recommendation may result in a reduced influx of oil into one or more wells (e.g., reduce influx of incompatible oil into one or more wells as in the context solid precipitation when different oil mixes). As still another example, a recommendation may result in identifying an entry point (e.g., drilling location) and path (e.g., drilling path) of a subsequent well adjacent to the well 420.

When a recommendation is generated, the controller 404 may also send the recommendation. In such a case, the controller 404 may use the communication module 507 and/or the application interface 526 to send the recommendation. A recommendation may be sent to one or more users 451 (including one or more associated user systems 455) automatically or on demand. A recommendation that is sent may have one or more of any of a number of formats. Such formats may include, but are not limited to, an email, a text message, an audio output, and a flashing indicating light. Yet another option may include generating a graphical representation using visual effects to depict one or more DHIP (e.g., SDHIP and/or EDHIP) of a well, one or more formation layers, one or more well, a recommendation, etc. Yet another option may include displaying the graphical representation in a graphical user interface. When step 789 is complete, the process proceeds to the END step.

FIG. 24 shows a gun barrel diagram 2498 of multiple wells in a subterranean formation having multiple formation layers for use with certain example embodiments. Referring to the description with respect to FIGS. 1 through 23 above, the gun barrel diagram 2498 of FIG. 24 shows a total of 9 horizontal wells 2420 viewed as a cross section slice through the lateral portion of each drilled well 2420. Specifically, the gun barrel diagram 2498 illustrates the subsurface vertical and lateral spacing of the horizontal sections of the wells 2420 within a development field. Listed in order of drilling/completion are well 2420-1, well 2420-2, well 2420-3, well 2420-4, well 2420-5, well 2420-6, well 2420-7, well 2420-8, and well 2420-9.

The subterranean formation 2410 shown in FIG. 24 has four formation layers 2439. Formation layer 2439-1 is located above formation layer 2439-2, which is located above formation layer 2439-3, which is located above formation layer 2439-4. Well 2420-3 is located in formation layer 2439-1 of the subterranean formation 2410. Well 2420-1 is located in formation layer 2439-2 of the subterranean formation 2410. Well 2420-4, well 2420-5, well 2420-6, well 2420-7, well 2420-8, and well 2420-9 are located in formation layer 2439-3 of the subterranean formation 2410. Well 2420-2 is located in formation layer 2439-4 of the subterranean formation 2410. There is a boundary 2493 between each pair of adjacent formation layers 2439. Specifically, boundary 2493-1 is between formation layer 2439-1 and formation layer 2439-2. Boundary 2493-2 is between formation layer 2439-2 and formation layer 2439-3. Boundary 2493-3 is between formation layer 2439-3 and formation layer 2439-4.

Using example embodiments, such as the process discussed above with respect to FIG. 7, it may be determined that well 2420-1 has a SDHIP 2463-1 and an EDHIP 2464-1 that exceeds the size of the SDHIP 2463-1. Well 2420-2 has a SDHIP 2463-2 and an EDHIP 2464-2 that exceeds the size of the SDHIP 2463-2. Well 2420-3 has a SDHIP 2463-3 and an EDHIP 2464-3 that exceeds the size of the SDHIP 2463-3. Well 2420-4 has a SDHIP 2463-4 and an EDHIP 2464-4 that exceeds the size of the SDHIP 2463-4. Well 2420-5 has a SDHIP 2463-5 and an EDHIP 2464-5 that exceeds the size of the SDHIP 2463-5. Well 2420-6 has a SDHIP 2463-6 and an EDHIP 2464-6 that exceeds the size of the SDHIP 2463-6. Well 2420-7 has a SDHIP 2463-7 and an EDHIP 2464-7 that exceeds the size of the SDHIP 2463-7. Well 2420-8 has a SDHIP 2463-8 and an EDHIP 2464-8 that exceeds the size of the SDHIP 2463-8. Well 2420-9 has a SDHIP 2463-9 and an EDHIP 2464-9 that exceeds the size of the SDHIP 2463-9.

Further, the gun barrel diagram 2498 shows that there is a direct overlap between the EDHIP 2464-1 of well 2420-1 and the EDHIP 2464-5 of well 2420-5. There is also a direct overlap between the EDHIP 2464-2 of well 2420-2 and the EDHIP 2464-4 of well 2420-4. There is also a direct overlap between the EDHIP 2464-3 of well 2420-3 and the EDHIP 2464-7 of well 2420-7. This information may be used to generate one or more recommendations about the operation of one or more of the wells 2420 and/or the development of a future well in the subterranean formation 2410.

In some cases, the process described with respect to FIG. 7 may be performed for a single well 420 in isolation. In other cases, the process described with respect to FIG. 7 may be performed for multiple wells 420 that share a subterranean formation (or one or more formation layers thereof) over time. For example, as after one well 420 is drilled and completed, the information gathered through the signatures of that well 420 may be used to inform future production practices for that well 420 and/or provide information as to the development and production of a future adjacent well 420. This process may repeat itself over time for multiples of adjacent wells 420, where the signatures (e.g., historical, present) generated from adjacent wells 420 is shared (e.g., compared, analyzed) with signatures (e.g., historical, present) generated from a current well 420.

Example embodiments may be used to provide systems and methods for estimating a DHIP for a well drilled into multiple formation layers. Example embodiments may provide a number of benefits. Such benefits may include, but are not limited to, optimizing well landing depth, optimizing well spacing, optimizing well completion design, optimizing well performance, optimizing hydraulic fracturing operations, optimizing sour gas injection operations, optimizing hydrocarbon gas injection and production wells, optimizing water flooding performance, optimizing steam flooding performance, optimizing polymer flooding performance, optimizing chemical flooding performance, extending the production life of a well (including both parent wells and child wells), configurability, and compliance with applicable industry standards and regulations. Optimizing may include improving depending on the context. Optimizing may include increasing depending on the context, such as, but not limited to, increasing hydrocarbon production. Optimizing may include decreasing or reducing depending on the context, such as, but not limited to, decreasing or reducing inefficiencies.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A method for estimating a stimulated drainage height in place for a well drilled into multiple formation layers, the method comprising:
analyzing a first plurality of samples to generate a first plurality of measurements of a plurality of parameters associated with the first plurality of samples, wherein the first plurality of samples originates from a first known range of depths in the well, and wherein the first plurality of samples is collected at a surface outside the well during a first period of time before production of the well;
generating a first plurality of signatures for the first plurality of samples using the first plurality of measurements;
analyzing a second plurality of samples to generate a second plurality of measurements of the plurality of parameters associated with the second plurality of samples, wherein the second plurality of samples originates from a second known range of depths in the well, wherein the second plurality of samples is collected at the surface outside the well during a second period of time during production of the well, and wherein the second plurality of samples comprises produced fluid;
generating a second plurality of signatures for the second plurality of samples using the second plurality of measurements;
comparing the second plurality of signatures and the first plurality of signatures;
generating a production allocation and a fluid correlation of the well based on comparing the second plurality of signatures and the first plurality of signatures, wherein the production allocation comprises a calibration matrix, and wherein the fluid correlation comprises a quality assurance and quality control matrix; and
generating an estimate of the stimulated drainage height in place for the well based on the production allocation and the fluid correlation of the well.

2. The method of claim 1, wherein the first plurality of samples comprises at least one of a group consisting of core samples, cuttings, downhole fluids, and produced fluids.

3. The method of claim 1, wherein comparing the second plurality of signatures with the first plurality of signatures comprises updating an algorithm used to generate a signature.

4. The method of claim 1, further comprising:
analyzing a third plurality of samples to generate a third plurality of measurements of the plurality of parameters associated with the third plurality of samples, wherein the third plurality of samples originates from a first known range of depths in an adjacent well, wherein the third plurality of samples is collected at the surface outside the adjacent well during a third period of time before production of the adjacent well, and wherein the second period of time precedes the third period of time;
generating a third plurality of signatures for the third plurality of samples using the third plurality of measurements;
comparing the third plurality of signatures, the first plurality of signatures, and the second plurality of signatures; and
generating a revised estimate of the stimulated drainage height in place for the well and an estimate of an extended drainage height in place for the well based on comparing the third plurality of signatures, the first plurality of signatures, and the second plurality of signatures.

5. The method of claim 4, further comprising:
analyzing a fourth plurality of samples to generate a fourth plurality of measurements of the plurality of parameters associated with the fourth plurality of samples, wherein the fourth plurality of samples originates from a second known range of depths in the adjacent well, and wherein the fourth plurality of samples is collected at the surface outside the adjacent well during a fourth period of time during production of the adjacent well;
generating a fourth plurality of signatures for the fourth plurality of samples using the fourth plurality of measurements;
comparing the fourth plurality of signatures, the first plurality of signatures, the second plurality of signatures, and the third plurality of signatures; and
generating a second revised estimate of the stimulated drainage height in place for the well and a revised estimate of the extended drainage height in place for the well based on comparing the fourth plurality of signatures, the first plurality of signatures, the second plurality of signatures, and the third plurality of signatures.

6. The method of claim 5, further comprising:
generating a second production allocation and a second fluid correlation of the adjacent well based on comparing the fourth plurality of signatures and the third plurality of signatures; and
generating an estimate of the stimulated drainage height in place for the adjacent well based on the second production allocation and the second fluid correlation of the adjacent well.

7. The method of claim 6, wherein generating the second production allocation and the second fluid correlation of the adjacent well is further based on comparing the fourth plurality of signatures, the first plurality of signatures, and the second plurality of signatures.

8. The method of claim 4, wherein the estimate of the extended drainage height in place is at least as large as the estimate of the stimulated drainage height in place.

9. The method of claim 1, wherein the stimulated drainage height in place traverses multiple formation layers.

10. The method of claim 1, further comprising:
obtaining the first plurality of samples prior to analyzing the first plurality of samples; and
obtaining the second plurality of samples prior to analyzing the second plurality of samples.

11. The method of claim 1, further comprising:
generating a recommendation for a field operation based on the estimate of the stimulated drainage height in place for the well.

12. The method of claim 11, wherein the recommendation results in a reduced influx of incompatible oil streams into the well.

13. The method of claim 11, wherein the recommendation results in identifying an entry point and drilling path of a subsequent well adjacent to the well.

14. The method of claim 1, wherein analyzing the first plurality of samples comprises using at least one of a group consisting of Fourier transformed infrared spectroscopy (FT-IR), x-ray fluorescence (XRF), gas chromatography, gas chromatograph-mass spectrometry, two dimensional gas chromatography (with FID and/or MS), three dimensional gas chromatography (with FID and/or MS), SARA analysis, biomarker analysis, stable carbon isotope analysis, stable sulfur isotope analysis, total sulfur analysis, elemental analysis, DNA sequencing for oil, UV-Vis spectroscopy, and transmission spectroscopy.

15. The method of claim 1, wherein analyzing the first plurality of samples comprises identifying and removing a subset of samples that include oil based mud.

16. The method of claim 1, wherein the stimulated drainage height in place for the well is used to estimate an extended fracture in place for the well.

17. The method of claim 1, wherein the production allocation is applied to oil source allocation for the well, one or more adjacent wells, or any combination thereof.

18. The method of claim 1, wherein the production allocation is applied to water source allocation for the well, one or more adjacent wells, or any combination thereof.

19. A data analysis system for estimating a stimulated drainage height in place for a well drilled into multiple formation layers, the system comprising:
a plurality of sensor devices for measuring a plurality of parameters associated with a plurality of samples; and
a controller communicably coupled to the plurality of sensor devices, wherein the controller is configured to:
analyze a first subset of a plurality of measurements taken by the plurality of sensor devices for a first subset of the plurality of samples, wherein the first subset of the plurality of samples originates from a first known range of depths in the well, and wherein the first subset of the plurality of samples is collected at a surface outside the well during a first period of time before production of the well;
generate a first plurality of signatures for the first subset of the plurality of samples using the first subset of the plurality of measurements;
analyze a second subset of the plurality of measurements taken by the plurality of sensor devices for a second subset of the plurality of samples collected, wherein the second subset of the plurality of samples originates from a second known range of depths in the well, and wherein the second subset of the plurality of samples is collected at a surface outside the well during a second period of time during production of the well;
generate a second plurality of signatures for the second subset of the plurality of samples using the second subset of the plurality of measurements;
compare the second plurality of signatures and the first plurality of signatures;
generate a production allocation and a fluid correlation of the well based on comparing the second plurality of signatures with the first plurality of signatures, wherein the production allocation comprises a calibration matrix, and wherein the fluid correlation comprises a quality assurance and quality control matrix; and
generate an estimate of the stimulated drainage height in place for the well based on the production allocation and the fluid correlation of the well.

20. A computer implemented method for estimating a stimulated drainage height in place for a well drilled into multiple formation layers, the computer implemented method comprising:
facilitate analyzing a first plurality of samples to generate a first plurality of measurements of a plurality of parameters associated with the first plurality of samples, wherein the first plurality of samples originates from a first known range of depths in the well, and wherein the first plurality of samples is collected at a surface outside the well during a first period of time before production of the well;
facilitate generating a first plurality of signatures for the first plurality of samples using the first plurality of measurements;
facilitate analyzing a second plurality of samples to generate a second plurality of measurements of the plurality of parameters associated with the second plurality of samples, wherein the second plurality of samples originates from a known depth in the well, wherein the second plurality of samples is collected at the surface outside the well during a second period of time during production of the well, and wherein the second plurality of samples comprises produced fluid;
facilitate generating a second plurality of signatures for the second plurality of samples using the second plurality of measurements;
facilitate comparing the second plurality of signatures and the first plurality of signatures;
facilitate generating a production allocation and a fluid correlation of the well based on comparing the second plurality of signatures and the first plurality of signatures, wherein the production allocation comprises a calibration matrix, and wherein the fluid correlation comprises a quality assurance and quality control matrix; and
facilitate generating an estimate of the stimulated drainage height in place for the well based on the production allocation and the fluid correlation of the well.
